(19) United States
(12) Patent Application Publication
(10) Pub. No.: US 2024/0180403 A1
(43) Pub. Date: Jun. 6, 2024

(54) ROTATABLE ENDOSCOPE SYSTEM HAVING A ROTATABLE INSERTION TUBE

(71) Applicant: JOSHI INNOVATIONS GmbH, Wurmlingen (DE)

(72) Inventors: Shirish Joshi, Wurmlingen (DE); Sourabh Ketkale, Tuttlingen (DE); Faisal Kalim, Munich (DE); Subhamoy Mandal, Heidelberg (DE)

(21) Appl. No.: 18/547,420

(22) PCT Filed: Feb. 16, 2022
(86) PCT No.: PCT/EP2022/053745

§ 371 Date: Aug. 22, 2023

(30) Foreign Application Priority Data
Feb. 23, 2021 (EP) 21158605.2

(51) Int. Cl. A61B 1/00; A61B 1/06; A61B 1/12; A61B 1/313

(52) U.S. Cl. CPC A61B 1/00147; A61B 1/00029; A61B 1/00128; A61B 1/0638; A61B 1/0669; A61B 1/126; A61B 1/3132

(57) ABSTRACT

An endoscope (1) is provided. The endoscope (1) comprises a fixed coupling member (2), and a rotatable shaft member (3) including a shaft (4) having a shaft axis (C) extending in an axial direction (d1), wherein the shaft (4) has a tip end (5) and a mounting end (6), wherein the shaft (4) is configured to transmit light from the mounting end (6) to the tip end (5) and to transmit image information from the tip end (5) to the mounting end (6). The coupling member (2) and the shaft member (3) are connected to each other such that the shaft member (3) is unlimitedly rotatable by 360° or more about the shaft axis (C) relative to the coupling member (2). Further, the shaft member (3) has a receiver (7) configured to receive energy.

The present invention relates to an endoscope.

Endoscopes are instruments for visualizing the interior of an object. In medical applications, an endoscope is used to examine and inspect the interior of the human body including internal organs, anatomical body cavities, and joints. A typical endoscope includes an elongated flexible or rigid insertion tube (the so-called shaft) within which a lens system is provided. At a tip end of the endoscope an optical system is provided. The image of the object being viewed by the optical system in a field of view is transmitted through the shaft for viewing by the user or for reception by a camera. Some endoscopes also have optical cables for illuminating the field of view with light supplied by an external light source.

Some endoscopes are designed to look at an angle to the axis of the shaft instead of in a straight line, such as 30 degrees. The angle in which the field of view may be inclined with respect to the axis of the shaft is defined between the axis of the shaft and a so-called direction of view. The direction of view may be a main direction in which the field of view is directed. The area under observation can be significantly increased by rotating the direction of view around the axis of shaft. The direction of view may be varied by a user, e.g. from 0 to 120 degrees with respect to the shaft axis. By combining inclining the direction of view and rotating the direction of view, a large area can be viewed without significant movement of the shaft of the endoscope. This makes the process safer as more movement of the shaft can cause more unintended injuries to critical structures in the body as well as the endoscope itself. Also, the endoscope is more likely to obstruct instruments which might be in use at same time.

Existing rigid endoscope technology generally consists of in-line systems where the tip end of the shaft is along the same axis as the eyepiece at the opposite end of the endoscope. For changing the direction of view, the shaft may be rotated with respect to a fixed body for holding the endoscope. However, this rotation is limited by the light transfer system due to fixedly connected optical cables. That is, during usage of the endoscope, there may be situations in which the user needs to further rotate the shaft in a specific direction but is hindered by a limit stop which is due to the internally connected optical fiber. In addition, if the optical fiber is externally connected to the rotatable shaft, there is the problem that the optical fiber is wounded around the shaft if the shaft is turned by 360°. Therefore, the handling of such endoscopes may be cumbersome.

WO 2006052769 A1 discloses a rotatable endoscope system having a rotatable insertion tube. The rotatable insertion tube enables the system to overcome ergonomic deficiencies found in previous endoscope systems by allowing the user to move the view field without rotating the entire endoscope.

However, the rotation of the shaft is still limited. Therefore, it is an object of the present invention to provide an endoscope having an improved operability to assist a user in handling the endoscope.

The present invention solves this problem with an endoscope having the features of claim 1. In particular, by combining a fully rotatable endoscope and an inclinable direction of view, it is possible to seamlessly follow an object (e.g. an endoscopic tool) with minimal movement of the shaft of the endoscope. Thus, potential injuries when automated systems are used may be avoided.

The endoscope may have a fixed coupling member. Further, the endoscope may have a rotatable shaft member including a shaft having a shaft axis extending in an axial direction, wherein the shaft has a tip end and a mounting end, wherein the shaft is configured to transmit light from the mounting end to the tip end and to transmit image information from the tip end to the mounting end. The coupling member and the shaft member may be connected to each other such that the shaft member is unlimitedly rotatable by 360° or more about the shaft axis relative to the coupling member. Further, the shaft member may have a receiver configured to receive energy.

The shaft member may be rotatable without limit stop, by at least 360°, preferably without limit, i.e. round and round any number of times. That is, a fully rotatable endoscope may be provided. Accordingly, the endoscope may have an increased operability because a user may readily adjust the direction of view defining the field of view independent of the current position of the shaft member relative to the coupling member. Hence, the endoscope may be also readily implemented in an automated system such as in a robotic system. Further, the Endoscope may have fewer parts to be rotated which may facilitate the handling of the endoscope. In addition, the shaft member (i.e. the rotatable element) may have a compact size because no external cables may be radially connected to the shaft member. Thus, the handling of the endoscope may be further improved. Preferably, if the shaft member can be rotated unlimitedly (i.e. without stop), objects to be visualized may be tracked more easily by a user in an uninterrupted manner. Further, the endoscope does not need to be moved in order to have an all-around view. Therefore, the space occupied by the endoscope during a surgery may be reduced. That is, compact size of an endoscope system is very important as operating space in surgery is limited. In addition, several instruments may be cluttering the operating field and extra moving cables reduce surgical freedom and therefore surgical results. In particular, the endoscope may be designed to look at an angle to the axis of the shaft (e.g. the angle may be 30° with respect to the shaft). The angle in which the field of view may be inclined with respect to the axis of the shaft is defined between the axis of the shaft and the direction of view. The area under observation may be significantly increased by being able to fully rotate the direction of view around the axis of shaft. In addition, the direction of view may be inclined with respect to the shaft axis e.g. in a range between 0 to 120 degrees.

Generally, the endoscope may provide an image of an object to be examined which is hard to access. Accordingly, the endoscope may have an eye piece (e.g. an ocular) in which a user may see the object (i.e. the image of the object). Alternatively or additionally, the endoscope may have an optical sensor (e.g. an image sensor). Then, an image of the object may be depicted on a display device. The optical sensor may be provided at the tip end of the shaft (e.g. a CCD-sensor) or in a housing connected to the coupling member. In case the sensor is provided at the tip end of the shaft, the image information may be electrical signals that are transmitted through the shaft. On the other hand, in case the sensor is provided within the housing, the image information may be light information that are transmitted through the shaft. The optical sensor may be provided within a rotatable part (e.g. the shaft member) of the endoscope. The power supply and/or the signal output may be transmitted from and/or to a stationary part (e.g. the coupling member). For example, the power supply may be realized via the receiver (e.g. wirelessly or via brush contacts). The signal transfer from the optical sensor may be realized wirelessly or via brush contacts.

Throughout the following description the following directions may be referred to:
an axial direction may correspond to an elongation direction of the shaft (e.g. the shaft axis may extend in the axial direction), and
a radial direction which may be a direction perpendicular to the axial direction.

The coupling member may be configured so as to allow the shaft member to be unlimitedly rotated about the shaft axis (e.g. to perform two revolutions about the shaft axis in one direction). Fixed in this case may mean that the fixed coupling member may be stationary with respect to the shaft member. In other words, the coupling member may be fixedly held by a user or by another supporting means, while the shaft member may be unlimitedly rotated about the shaft axis. The coupling member (also referred to as coupling attachment) may be a coupling mechanism connecting the shaft member and a fixed body (e.g. the housing). The fixed body may include an eye piece and/or a connection an external camera or viewer system to the endoscope. In addition, the fixed body may include a photo sensor. Accordingly, the fixed body may have an image information passage configured to transmit the image information. Further, the fixed body may have a gripping portion at its outer surface such that a user may ergonomically handle the endoscope.

The coupling member may have an adjustment means for adjusting the focus of the endoscope (i.e. of the image of the object). Preferably, the adjustment means is a focusing ring. Accordingly, by rotating the focusing ring relative to the coupling member, the user may adjust the focus point of the image. Therefore, the endoscope may offer the possibility of adjusting the image (which may be based on the image information) to the optimum sharpness, even for spectacle wearers, by means of the focusing ring. Further, an eyepiece may be arranged or arrangeable at the coupling member. In addition, a coupling device may be connected to the coupling member. The coupling device may be a fixed (i.e. stationary) body with respect to the shaft member. An image sensor may be provided within the coupling device or may be attached to the coupling device so as to receive the image information transmitted by the shaft. The coupling member and the coupling device may be formed as an integral element.

In an embodiment the coupling member has an adjustment means for adjusting the focus of the endoscope automatically and/or manually. Adjusting the focus manually may mean that the focus may be adjusted by a user via the above focus ring, for example. Automatically adjusting the focus may mean that the adjustment means automatically adjust the focus triggered by the user or an algorithm. The user may designate a point within the field of view to be in focus, and the adjustment means may adjust the focus accordingly to bring that point into focus. Alternatively, the adjustment means may be controlled such that the center of the field of view is brought into focus. Further, the adjustment means may be controlled such that a tool within the field of view is always brought into focus. In any case, the adjustment means may be controlled by a control unit configured to process data. The desired focus point may be input data to the control unit and commands to the adjustment means may be output data. Accordingly, the algorithm may be executed on the control unit and process the input data to the output data. The adjusting means may be operated by an actuator (e.g., a motor). That is, the commands may operate the actuator which may control the adjusting means. Alternatively, the adjusting means may comprise an electrically tunable-focusing liquid lens. Such tunable-focusing liquid lens may adjust the focus point by applying different voltages to the liquid. In this case, the adjusting means may adjust the focus by applying a specific voltage to the lens based on the desired point within the field of view which should be focused. In any case, automatic focus adjustment can result in precisely adjustable focus so that even moving objects always remain in focus.

The shaft member may comprise the shaft and a mounting base, wherein the shaft may be mounted to the mounting base so as to rotate together with the mounting base about the shaft axis. The mounting base may be formed in a cylindrical shape having a mounting base axial axis which is aligned with the shaft axis. For example, the receiver may be accommodated within the mounting base. The shaft and the mounting base may be formed as an integral element. Further, the shaft member may be axially symmetrical with respect to the shaft axis. Accordingly, the weight distribution is optimally balanced so as to increase the operability of the endoscope.

The shaft may be formed as a hollow rod like element. For example, the shaft may be an elongated and cylindrical body. Further, the shaft may be flexible or rigid or a combination of both. The flexible shaft may allow an advantageous use of the endoscope in Laparoscopy, gastroscopy, colonoscopy, bronchoscopy and arthroscopy. Further, an endoscope having the flexible shaft may also be used as a cardiac catheter. That is, the shaft may follow a contorted passage so as to continuously investigate the passage and/or objects the passage leads to. The image information may be transferred through the shaft via one optical fiber or a plurality of optical fibers.

The rigid shaft may allow a use of the endoscope as a technical borescope and, in the medical field, as an arthroscope and cystoscope, for example. The shaft may include a rod lens system. Within the rigid shaft, image information may be guided through the rod lens system by rod lenses made of quartz glass, for example, and refracted at air lenses between the rod lenses. This very light-intense design (i.e. a design that absorbs only very little light) allows for smaller lens diameters and thus for smaller shaft diameters.

In any case, the shaft may include an image information guide in the form of an image guide or an electrical conductor. The image guide may transmit image information in the form of light information to a sensor provided within the endoscope or to an external sensor. The electrical conductor may transmit digital image information as data generated by an image sensor provided at the tip end of the shaft.

Further, the shaft may include a light guide for guiding light from the mounting end to the tip end. The light may be introduced into the light guide at the mounting base. For example, the light may be received from a light source (e.g. a LED) provided in the mounting base or from another light source. In case the light source is provided within the mounting base, the light source may be energized by the energy received by the receiver. In this case, the receiver may wirelessly receive energy from an external energy source (more details will follow below). Alternatively, the receiver may wirelessly receive light from an external light source. In this case the light may be transmitted to the receiver without a tethered connection (more details will follow below). Therefore, the boundless/unlimited rotation of the shaft member may be possible.

The light may be emitted by the tip end of the shaft so as to illuminate the field of view. Accordingly, an examination and/or inspection may be possible even in dark environments.

That is, the shaft may be configured to transmit the light for illumination in one direction along the shaft axis and the image information in the opposite direction along the shaft axis. Accordingly, both illuminating the field of view and acquiring image information of the field of view may be realized by one single shaft. In other words, the shaft may be configured to transmit light in a bidirectional manner. Therefore, the endoscope may be compact.

The tip end may be also referred to as a distal end of the shaft. The tip end may have an opening through which light may enter and exit the interior of the shaft. The opening may define a surface which may be inclined with respect to the shaft axis by more or less than 90°. That is, the surface may be not perpendicular to the shaft axis. Accordingly, the tip end may be open to a side of the shaft at least partly towards the radial direction.

At the opening an optical system (e.g. a mirror system and/or a prism) may be provided for receiving image information of an object to be imaged. The image information may be light rays reflected by the object. Therefore, the tip end of the shaft may be configured to receive image information from a specific direction (i.e. from a field of view) inclined with respect to the shaft axis. Further, the optical system may be configured so as to redirect the image information of the object into the image information guide, that is, along the orientation of the shaft axis. Subsequently, the image information may be transmitted through the shaft (i.e. via the image information guide) along the shaft axis to the mounting end. As a result, by rotating the shaft about the shaft axis, the direction of view (and thus the field of view) may be rotated so as to provide an all-around view about the tip end. In addition, the optical system may be configured to adjust the angle between the direction of view and the shaft axis so as to change the direction of view. Accordingly, the direction of view may be individually adjusted without moving the shaft so as to holistically image objects existing near the tip end. Thereby, the area that may be imaged by the endoscope without transversely and/or longitudinally moving the endoscope may be enlarged.

Moreover, the tip end of the shaft may have at least one illumination opening for emitting light transmitted from the mounting end to the tip end. Preferably, the illumination opening is provided adjacent to the optical system so as to illuminate the field of view. That is, the illumination opening may be preferably directed towards the same region as the optical system. Preferably, the illumination opening is part of the optical system. For example, the tip end may have four illumination openings so as to provide a sufficient illumination of the field of view.

The mounting end of the shaft may be also referred to as a proximal end of the shaft. The mounting end may be the part of the shaft with which the shaft is mounted to the mounting base. The shaft and the mounting base may be an integral element. Thereby, the maintenance of the endoscope may be facilitated (e.g. no retightening of the shaft and the mounting base may be necessary). Alternatively, the shaft may be detachable from the mounting end. Hence, different shafts may be used with the same mounting base. Accordingly, the endoscope may be adapted to different applications by providing different shafts (e.g. different length of shafts or different optical properties). For example, the shaft may be connected to the mounting base by screwing.

For transmitting the light through the shaft via light guides, glass fibers may be used. However, there may be also provided light guides that can conduct the light using a gel as a transport medium. Gel light guides or liquid light guides offer a stronger light output, which is particularly advantageous for illuminating a large field of view and for digital endoscopy. Liquid light guides may be better suited for transmitting UV or IR light than fiber optics. Gel light guides or liquid light guides may be a bit more awkward to use because they are not as flexible than fiber optic light guides.

The image information (e.g. the reflected light rays from the object to be examined) may be guided through the shaft via the image information guide that may be made up of a plurality of (e.g. many thousands of) individual glass fibers, with a diameter of 7 to 10 μm, for example. In this case a resolution of the image may be 3,000 to 42,000 or 75×45 to 240×180 pixels, depending on the diameter of the image guide. That is, brightness information and color information for one pixel may be transmitted per fiber. Preferably, the image information guide may be centered on the shaft axis. That is, a central axis of the image information guide may be positioned on the shaft axis. The image information guide may be circumferentially surrounded by the light guide.

Alternatively, the image information may be transmitted via an electrical conductor in case an image sensor is provided at the tip end of the shaft. The electrical conductor may be made of metal. The electrical conductor may transmit the digital image information to the mounting end of the shaft or to the mounting base. Within the mounting base an image transmitter may be provided which may be configured to transmit the image information to a processing system. Preferably, the image transmitter is configured to wirelessly transmit the digital image information.

The shaft member and the coupling member are rotatably connected to each other. Preferably, a radial bearing is provided so as to allow the rotation of the shaft member with respect to the coupling member. That is, in any other degree of freedom a movement of the shaft member with respect to the coupling member may be prevented. Therefore, the endoscope may be securely handled by the user. The bearing may be an anti-friction bearing so as to allow a smooth and easy rotation of the shaft member, such as a ball bearing, a cylindrical roller bearing, a needle roller bearing or a tapered roller bearing.

In this case unlimitedly (i.e. continuously) rotatable by 360° or more may mean that the shaft member may be indefinitely rotatable about the shaft axis with respect to the coupling member. In other words, the shaft member may be rotated several revolutions in series in the same direction. That is, there is no stop provided that may hinder the unlimited or continuous rotation of the shaft member with respect to the coupling member. In addition, there may be no connection by optical fibers or electrical wires between the shaft member and the coupling member. Thus, the handling of the endoscope may be significantly facilitated. In addition, an automized handling of the endoscope may be easily implemented.

The receiver may be a device that may receive energy without being tethered (e.g. being mechanically connected) to an energy source. Therefore, no cable or other energy transmitting element may physically reach from the coupling member to the shaft member. Accordingly, the shaft member and the coupling member may be freely rotatable with respect to each other.

The energy transmitted to the receiver may be in the form of electromagnetic energy or waves such as light or lower frequency electromagnetic energy. The transmission of energy to the receiver may be realized by inductive coupling, resonant inductive coupling, capacitive coupling, magnetodynamic coupling, transmission of microwaves or light waves. In case of inductive coupling the receiver may be formed as a wire coil. In case of resonant inductive coupling the receiver may be formed as a tuned wire coil or a lumped-element resonator. In case of capacitive coupling the receiver may be formed as a metal plate electrode. In case of magnetodynamic coupling, the receiver may be formed as a rotating magnet. In case of transmission of microwaves, the receiver may be formed as an antenna comprising a parabolic dish, phased array or a rectennas rotating magnet. In case of light wave transmission, the receiver may be formed as a photocell. In any of the above cases the receiver may receive energy which may be transformed into electrical energy by a circuitry provided within the shaft member. The electrical energy may be supplied to a light source within the shaft member so as to generate light. The light may then be transferred to the light guide provided within the shaft (further details will follow below).

Further, in case of light wave transmission, the receiver may be formed as a laser receiver or a light transmitting surface. Accordingly, the receiver may be configured to directly receive light which may be then guided to the light guide within the shaft. In this case, the receiver may be configured to transmit the light to the light guide provided within the shaft.

Further, a transmitter may be provided which may be configured to transmit one of the above forms of energy to the receiver. For example, such transmitter may be provided within the coupling member.

That is, between the rotatable shaft member and the stationary coupling member a transfer interface may be formed. Via the transfer interface at least energy may be transferred from the coupling member to the shaft member. In some embodiments, energy may be transferred from the coupling member to the shaft member and image information may be transferred from the shaft member to the coupling member. That is, the transfer interface may transfer energy and image information in a bidirectional manner. In any case, there is preferably no mechanical connection in the interface such that the shaft member may be freely rotatable with respect to the coupling member. The transfer interface may be formed by the receiver of the shaft member and by a transmitter provided within the coupling member. Further, the transfer interface may be provided on an extension of the shaft axis. Accordingly, the endoscope may be compact.

In an embodiment, the coupling member has an image information passage configured to transfer the image information from the shaft member in the axial direction. The image information passage of the coupling member may be aligned to the shaft such that the image information may be transmitted from the shaft to the coupling member independent of the rotational position of the shaft. That is, the image information passage may be aligned to the image information guide of the shaft. For example, the image information passage may be positioned on an extension of the shaft axis so as to be symmetrical to said axis. Accordingly, the image information may be appropriately transmitted from the shaft member to the coupling member independent of the position of the shaft member with respect to the coupling member.

Further, the coupling member may include a lens system, preferably a rod lens system, so as to further transmit the image information to an eye piece and/or to an image sensor. Accordingly, the image information may be transmitted without excessively absorbing light (i.e. information).

In an embodiment, the coupling member has a transmitter configured to input the energy to the receiver in the axial direction or radial direction. As mentioned above, the transmitter may be configured so as to correspond the receiver. Further, the receiver and the transmitter may face each other in the axial direction. That is, the transfer interface may define a rotatable surface (at the shaft member) and a stationary surface (at the coupling member) which may be formed so as to be perpendicular to the axial direction. The receiver and the transmitter may have an annular shape. Accordingly, the image information passage may pass through a center of the receiver and the transmitter. In other words, the receiver and the transmitter may circumferentially surround the image information passage. Therefore, the energy (e.g. light, electromagnetic field or waves) may be sufficiently transmitted from the transmitter to the receiver independent of the relative position of the shaft member and the coupling member. At the same time, the image information may be sufficiently transmitted from the shaft member to the coupling member.

In an embodiment, the coupling member has at least one port for receiving light from a light source, and wherein the coupling member is configured to transmit energy in the form of light to the receiver of the shaft member in the axial direction. An optical waveguide may be attached or attachable to the port so as to transfer light to the coupling member. That is, the waveguide which may be an optical fiber, may be connected to the fixed and stationary part (i.e. the coupling member) of the endoscope. Therefore, there is no such problem like winding the waveguide about the shaft when rotating the shaft. Within the coupling member the light may be further transmitted to the receiver. In more detail, the transmitter (stationary part) may send the light via the transfer interface to the receiver (moving part).

The light source may be an external light source (i.e. may be provided within a separate housing). In particular, xenon lamps may be used as a light source. An external light source may exhibit a power of about 80 Watts. Especially, if the image sensor is provided at the tip end of the shaft, a powerful light source may be necessary to sufficiently illuminate the field of view. However, xenon lamps may generate an enormous amount of heat during operation, most of which is caused by the infrared component of the light source spectrum. Therefore, the IR component may be prevented from reaching the tip end of the shaft in order to prevent an excessive heating of the shaft. Therefore, the light source may be adjustable in light intensity and/or cooled by a fan. Further, the infrared radiation may be removed or reduced from the light spectrum by dichroic concave mirrors and/or by heat protection filters in front of the light guide (i.e. before the light enters into the light guide of the shaft). The dichroic concave mirrors and/or the heat protection filters may be provided within the coupling member so as to reduce the IR component. Therefore, the endoscope may deliver an improved visualization of the object to be examined while an excessive heating of the endoscope may be prevented.

In an embodiment, the at least one port is connected to the coupling member such that the light is inputted into the coupling member in a radial direction perpendicular to the axial direction, and wherein the coupling member is configured to redirect the light so as to be transmitted to the receiver in the axial direction, preferably by a partly reflective mirror array or a micro mirror array.

That is, the coupling member may be configured to redirect the light supplied from an external light source and inputted into the coupling member in the radial direction, along an extension of the shaft axis. That is, the light may be redirected only once within the endoscope. Therefore, the light may be directly inputted into the light guide without being redirected several times. The redirection of the light may be done by reflective mirrors (e.g. micro-mirrors) or prism arrays. The micro mirror may be formed by grooves in an optical substrate (e.g. the optical fiber). The micro-mirror grooves may be spaced apart so as to transmit a collimated beam with symmetrical beam transport. Therefore, the redirected light may be aligned such that the light rays may be essentially parallel to each other. Preferably, the light rays may be redirected by the coupling member such that they are parallel to the axial direction.

The redirection of the light may be realized by a distributor. The distributor may be formed of a light-transmissive substrate. The distributor may have at least one inlet connected to the at least one port and one outlet connected to the transmitter of the coupling member. The shape of the outlet of the distributor may correspond to the shape of the transmitter (e.g. may have an annular shape around an extension of the shaft axis). Further, the distributor may have the partly reflective mirrors or prism arrays so as to evenly distribute and transmit the light at the outlet of the distributor. The partly reflective mirrors or prism arrays may be provided so as to extend over the whole cross section of the light transmissive substrate. Alternatively, the distributor may have the micro-mirror arrays so as to evenly distribute and transmit the light at the outlet of the distributor. The micro-mirror arrays may only partly extend over the cross section of the light transmissive substrate. Preferably, the distributor may align the light rays so as to be essentially parallel to the axial direction.

The alignment of incoming and transmitted light rays may reduce stray reflections which may reduce distortions. As a result, the efficiency of light transmission may be increased. Further details of the mirrors (micro-mirror) or prism arrays could be found in the paper of Miaomiao Xu and Hong Hua “Methods of optimizing and evaluating geometrical lightguides with microstructure mirrors for augmented reality displays” Opt. Express 27, 5523-5543 (2019) which content is incorporated herein by reference.

In an embodiment, the coupling member has a transmitter, wherein the transmitter of the coupling member and the receiver of the shaft member are each formed as a light transfer surface, wherein the receiver and the transmitter are facing each other in the axial direction and are configured to transfer the light, wherein the receiver and the transmitter preferably have a circular shape.

In another embodiment, the receiver and the transmitter are arranged circumferentially on an extension of the shaft axis and may have a through hole for passing the image information passage through the receiver and the transmitter. In other words, the receiver and the transmitter may be formed as mating surfaces so as to allow a relative rotation between the shaft member and the coupling member. Accordingly, the receiver and the transmitter may have an annular shape.

In a useful embodiment, the transmitter and receiver are made of glass, transparent plastic or glass fiber.

In addition, the transmitter and receiver may be made of acrylic glass. That is, the receiver and the transmitter may be made of a light-transmissive material so as to reduce refraction and/or reflection. As a result, a contactless transmission of light may be possible. Hence, a relative rotation of the shaft member and the coupling member may be readily possible without limitations.

In an embodiment, the transmitter and the receiver are spaced apart from each other in the axial direction so as to form a gap between the receiver and the transmitter, wherein a transparent fluid is provided within the gap. The gap may be between 0.5 μm and 500 μm, preferably between 3 μm and 20 μm.

The gap may allow a relative movement (e.g. a rotation) of the shaft member and the coupling member without the danger of damaging either the receiver or the transmitter or both due an excessive occurrence of friction. At the same time the fluid may prevent that the light rays are excessively refracted due to being transmitted from a solid medium to a gaseous medium. That is, the fluid provided within the gap may essentially maintain the direction of the light rays (e.g. parallel to the axial direction). Preferably, the fluid has a similar refraction index than the transmitter and/or the receiver. Therefore, the endoscope may exhibit an improved durability and a high light transfer efficiency. The fluid may be gas or a high transmission transparent liquid medium. Especially in the latter case, the light could be transmitted from the coupling member to the shaft member without being excessively refracted at the phase change between air and solid medium. Therefore, the efficiency of light transfer may be further increased. Alternatively, a vacuum may be provided within the gap. Accordingly, the shaft member and the coupling member may comprise a sealing element so as to maintain the vacuum within the gap. Alternatively, the shaft member may be in direct contact with the coupling member. In particular, the receiver may be in direct contact with the transmitter. In this case there may be no gap between the shaft member and the coupling member.

In an embodiment, the shaft member includes at least one light source, preferably a LED, that is operated by electric energy received by the receiver. The light source may comprise a plurality of lamps arranged circumferentially around the extension of the shaft axis. The lamps may be arranged within the mounting base of the shaft member. Accordingly, if the shaft member is rotated, the lamps may be also rotated. Therefore, no transmission of light from a non-moving element to a moving element is necessary. The lamps may be connected to the light guide of the shaft by an optical fiber, for example. In addition, in order to transform the energy received by the receiver into electrical energy, the shaft member may include circuitry. In particular, the shaft member may include a rectifying circuit.

The lamps provided within the shaft member may have a power of about 10 Watts. It is to be noted that the internal lamps may have less power than an external light source. However, the light generated by an internal light source does not have to be transmitted through a flexible cable to the endoscope and may thus not be attenuated due to reflection and/or refraction like the light from the external light source. Therefore, the internal lamps may have less power while nevertheless providing sufficient light so as to illuminate the field of view. Hence, the overall system may have an increased efficiency by reducing the losses during light transfer.

In an embodiment, the energy is inputted to the shaft member by a wireless power supply, preferably by inductive coupling, resonant inductive coupling or by magnetodynamic coupling. The power supply or energy supply may be an external transmitter transmitting energy to the receiver. The external transmitter may be a coil through which an alternating current may be conducted such that the coil creates an oscillating magnetic field by Ampere's law. The receiver may be a coil through which the magnetic field passes to as to induce an alternating electromotive force (voltage) by Faraday's law of induction, which creates an alternating current in the receiver. Accordingly, the endoscope may have a low weight because the transmitter may not be provided within the endoscope. Therefore, the operability of the endoscope may be increased. The external power supply may be provided near to the site of operation of the endoscope so as to ensure a sufficient power supply to the endoscope. Therefore, the power supply may be provided in a patient bed on which the patient to be examined may be positioned.

In an embodiment, the coupling member has a transmitter, wherein the transmitter of the coupling member is formed as a transmission coil and the receiver of the shaft member is formed as receiving coil, wherein the receiver and the transmitter are facing each other in the axial direction and are configured to transfer the energy. That is, similar to the arrangement of the light transfer surface of another embodiment of the present invention, in this embodiment the receiving coil and the transmitting coil may be arranged so as to face each other in the axial direction. That is, the coils may be arranged circumferentially around an extension of the shaft axis. Therefore, the image information passage may pass through a center of the coils. Further, the receiving coil and the transmitting coil may be arranged close to each other in order to ensure a sufficient energy transmission.

In another embodiment, the energy is inputted to the shaft member via brush contacts between the rotatable shaft member and the fixed coupling member in the form of electrical energy. In this embodiment, the shaft member may have a cylindrical contact portion. The contact portion may be arranged at the mounting base and may extend in the axial direction. The contact portion may be arranged at an opposite side at the mounting base with respect to the shaft. The contact portion may have electrical contacts as the receiver. The electrical contacts may be provided on a circumference surface of the contact portion facing towards the radial direction. The contacts may be arranged circumferentially at the contact portion. The coupling member may have a receptacle for receiving the contact portion. The receptacle may have contacts corresponding to the contacts of the contact portion. The contacts of the receptacle may be biased by a spring such that a contact between the contacts of the receptacle and the contacts of the contact portion may be ensured. Further, the receptacle may have a bearing for rotatably supporting the shaft member via the contact portion.

In an embodiment, the shaft member has different light sources each having a different wavelength. Accordingly, the illumination of the field of view may be improved by combining different wavelengths. For example, light having the UV or IR wavelength may be used to make differences between substance visible. Therefore, the endoscope may allow improved accuracy during surgery.

In an embodiment the endoscope comprises a cleaning device configured to clean the tip end of the shaft by wiping and/or vibrating. The tip end may comprise a lens through which the image information may pass. The lens may cover an opening of the shaft at the tip end of the shaft. In other words, the tip end (e.g., the lens) may be within the human body during an examination procedure. Accordingly, the tip end may be contaminated with water, blood or other foreign particles. The cleaning device may be configured to remove such contamination so as to allow the image information to enter smoothly into the shaft. As a result, the endoscope may deliver optimal image quality without the need to extract the endoscope out of the human body to clean it. The cleaning device may be configured to clean the tip end by wiping. That is, the cleaning device may comprise a wiper configured to wipe off water, blood or other foreign particles from the tip end of the shaft (e.g., from a lens provided at the tip end). In this case, the wiper may be configured in an easy way. Alternatively, the wiper may execute a rotational movement or a back and forth (i.e., bidirectional) movement. In this case, even small tip ends may be cleaned from undesired substances. The wiper may be a flexible element. Hence, scratching a lens or other elements provided at the tip end of the shaft may be avoided. Further, a prism may be provided at the tip end of the shaft. In particular, the prism may be configured to define a direction of view. Accordingly, the prism may be moved to adjust the direction of view. The flexible element may be fixed against the prism or against a mechanism configured to move the prism. When the prism is moved, the flexible element may perform a wiping or brush movement at the end of the shaft. The flexible element may be fixed or movable relative to the prism. The action of moving the prism may be achieved either by moving the whole endoscope or only by moving the prism. The flexible element used for cleaning may be mounted either on the endoscope or on a tube or trocar used to guide the endoscope in to the surgical area (further details will follow below). Hence, if the viewing direction of the endoscope is adjusted, the flexible element may be automatically activated so as to clean the tip end of the shaft. In this way, the image information can be introduced into the shaft undisturbed by foreign matter.

Further, the cleaning device may clean the tip end of the shaft by vibrating. That is, the cleaning device may be configured to induce vibration of the tip end so that unwanted substances are shaken off the tip end of the shaft. In this case the cleaning device may include a piezo element configured to generate vibrations. The vibrations may be transferred to the tip end of the shaft. Especially, the vibrations may be generated by vibrating the prism. In other words, the cleaning device may be configured to vibrate the prism. The prism may then transmit the vibrations the tip end of the shaft so as to remove foreign matter. Vibrating the prism can cause a greater amount of material to vibrate, resulting in a more intense vibration of the tip end. As a result, no moveable parts have to be provided at an outside of the shaft. Thus, the endoscope may be compact. The cleaning device may oscillate with 15,000 to 35,000 oscillations per minute, preferably with 25,000 to 32,000 oscillations per minute. The first range allows to provoke a vibration at which even sticky matter may be removed from the tip end. In the latter range sound wave with a frequency of approx. 250 to 270 Hz may be emitted. It was found that in this range the handling of the endoscope is not negatively influenced (due to the vibrating tip end of the shaft) and the unwanted subject may though be removed.

Moreover, the cleaning device may have a combination of a wiper and a vibrator so as to remove unwanted substances from the tip end by both wiping and vibrating. Accordingly, any unwanted substance may be efficiently removed from the tip end.

In an embodiment the endoscope comprises a cleaning fluid supply device configured to transfer a cleaning fluid and/or gas to the tip end of the shaft and to eject the cleaning fluid and/or gas toward the tip end of the shaft.

The cleaning fluid supply device may be a hose or tube configured to transfer fluid such as water to the tip end of the shaft. The cleaning fluid supply device may be routed inside the shaft or outside the shaft. Further, the cleaning fluid supply device may have an ejection tip which may be configured to eject the fluid toward the tip end. The ejection tip may be formed as a nozzle. Hence, the fluid may be precisely ejected toward the tip end to remove unwanted matter from the tip end (e.g., from a lens provided at the tip end). The cleaning fluid supply device may be provided additionally to the cleaning device. In this case, unwanted subject matter may be satisfactorily removed from the tip end. In addition, the cleaning fluid supply device may be configured to clean the prism provided in or within the shaft. The cleaning fluid supply device may be provided at the shaft so as to not rotate with the shaft. In other words, the cleaning fluid supply device may be fixed to the coupling member. Accordingly, the shaft may be freely rotatable with respect to the fluid supply device. Therefore, no hose, port or other means connected to the fluid supply device may hinder the rotation of the shaft. The prism may be moved in such way as to achieve best alignment to spray the cleaning fluid. In other words, the prism may be moveable so as to be oriented towards the cleaning fluid supply device (e.g., towards the ejection tip). Further, the cleaning fluid supply device may be configured to transfer a gas to the tip end of the endoscope. The gas may be subsequently used to remove or wipe off excess cleaning liquid. The gas may be sprayed towards the tip end of the endoscope. The cleaning fluid supply device may be designed to spray fluid on the cleaning device (e.g. the flexible wiper) to remove accumulated dirt or fluid from the cleaning device. In this way, the longevity and effectiveness of the cleaning device may be ensured. The cleaning fluid may be a liquid, a gas or a combination thereof.

In an embodiment the endoscope comprises a time-of-flight sensor. Using the time-of-flight sensor (ToF-sensor) a depth of different objects in the image may be estimated. The ToF sensor may be used along with beam splitter for imaging objects in higher resolution. The endoscope may be operated in such way that the area under observation is observed from multiple positions to create a depth map of the same. For example, the endoscope may continuously or intermittently move in or out by few centimeters, for example. Counter zooming may be applied by the adjusting means when the endoscope is moved so that the operator is undisturbed by this movement. In this way, in addition to the 2D image information, a depth (i.e., a z-coordinate) may be obtained.

In an embodiment, the coupling member comprises an actuator for rotating the shaft member relative to the coupling member. That is, a gear may be provided so as to rotate the shaft member by an actuator. The power train may extend on an extension of the shaft axis. That is, the image information passage may circumferentially surround the power train. Alternatively, the power train may be positioned outward in the radial direction from the image information passage and may comprise a threaded rod which may be engaged with a corresponding gear at the shaft member. Accordingly, the endoscope may be automatically operated by a robot or by remote control. In addition, the endoscope may be operated in highly accurate manner. The actuator may be part of a robotic system configured to perform automated or semi-automated surgical procedures. In other words, the endoscope can advantageously be operated by a robot. In particular, the unrestricted rotatability of the shaft relative to the coupling element facilitates the use of the endoscope in conjunction with an automated robotic system.

In a useful embodiment, the coupling member comprises a sensor for detecting the position of the shaft member with respect to the coupling member.

The sensor may determine the relative position of the shaft member and the coupling member. Preferably, the relative position may be determined using a contactless sensor system. For example, the position may be determined, by a magnet wheel provided at the shaft member and a hall sensor provided at the coupling member. The position data may be transferred to an automated operation system so as to accurately control the endoscope. Therefore, a region of interest may be accurately reached and visualized by the endoscope. In addition, the information of the relative position of the shaft member with respect to the coupling member may be useful in an automated operation of the endoscope and in remote operation of the endoscope.

According to a further aspect of the present invention an imaging system is provided, comprising: an optical channel configured to transfer light, a first sensor configured to generate first image data by imaging an object along a first optical path, and a second sensor configured to generate second image data by imaging the object along a second optical path, wherein the first sensor and the second sensor are focus shifted, and wherein the first optical path and the second optical path are guided at least partly through the optical channel. The imaging system may be provided within one of the above endoscopes (e.g., within the coupling member). The optical channel may extend along the shaft axis of the shaft within the shaft. The imaging system may be the part of the endoscope responsible for gather the image information and to preferably evaluate and/or to process the image information.

The imaging system may be an optical imaging system and may be used to create image data of an object using the first sensor and the second sensor. That is, the first image data and the second image data may image the same part of the object from the same viewing angle. This is achieved by providing a dual sensor imaging system that may generate first and second image data in quick succession or simultaneously. The imaging data preferably comprises a sequence of images, e.g. a video stream. Further, since the first optical path and the second optical path are guided through the same optical channel, the direction in which the object is imaged (i.e. the viewing angle) of the first sensor and the second sensor is the same. As a result, the first image data and the second image data are automatically registered to each other so as to perfectly overlap with one another without the need to execute any additional alignment or registration process. Since the first sensor and the second sensor are focus shifted, the first image data has a different focus point (i.e. a point at which the object is sharply depicted by the image data) as compared to the second image data. That is, the first sensor may be focused to a point having a first distance d1 from a distal end of the optical channel and the second sensor may be focused to a further point having a second distance d2 from the distal end of the optical channel. The difference of the first distance d1 and of the second distance d2 may be the focus shift of the two sensors. The focus shift may be determined by the hardware of the imaging system. In more detail, the focus shift may be provided by the arrangement of the sensors within the imaging system and/or by an optical systems within the imaging system. Accordingly, the focal length and/or the optical paths may be shortened or extended by a specific arrangement of the sensors or by the optical system (to be explained in more detail below).

The imaging system may be provided within a laparoscope which may be used to provide inevitable information for diagnosing and treatment related to internal organs of the human or animal body. In addition, the laparoscope may be used in maintenance of large machinery, e.g. to check whether gears that are difficult to access have to be exchanged or not, without demounting the whole machinery. The sensors of the imaging system may be accommodated within a camera head of the laparoscope such that a camera head may be provided having a dual sensor, wherein each sensor has its own focus. In other words, the sensors may be housed solely in the camera head so that there is no sensor in the shaft. Accordingly, the shaft may be made slim so that it may be operated without problems in narrow cavities, for example. Therefore, the sensors may be outside the cavity to be inspected during operation. Among other things, this leads to the fact that the weight distribution of the endoscope may be advantageously modified so that the shaft has a relatively low weight compared to the camera head. Since the endoscope is held at or near the camera head by the user or robot, the operation of the endoscope may be further simplified. That is, each sensor may have its own focus point such that the sensors are focus shifted with respect to each other. For example, each sensor may have its own focal length. That is, the focal length of the sensors may be different. Further, the laparoscope may have a shaft protruding from the camera head, that may be brought in the vicinity of the object to be imaged. That is, the shaft may be at least partly inserted into a human body or into a narrow cavity, for example, and wherein the shaft may comprise the optical channel. The shaft may be connected to the camera head at a proximal end thereof. The camera head may be removed from the shaft. In other words, the sensors may be separated from the shaft. Accordingly, the sensors may be easily replaced, changed, repaired, maintained, etc. without having to disassemble the shaft itself. This means that the shaft may continue to be used, while only the camera head may be replaced. The distal end of the shaft may be facing the object to be imaged. The shaft as well as the optical channel may be configured to be at least partly flexible. Furthermore, the shaft may be controllably moveable (e.g. controllably bendable so as to redirect the distal end of the shaft). As a result, the laparoscope may be adapted to any environment in which it is to be used and may reach regions located behind other objects. Within the shaft the optical channel may be provided (e.g. in the form of a wave guide) which is configured to guide the first optical path and the second optical path from the proximal end of the shaft to the distal end of the shaft independent of the flexion of the shaft. In addition, at the distal end and/or at the proximal end of the shaft, an optical device (e.g. a lens or an array of lenses) may be provided, respectively, in order to appropriately guide the first optical path and the second optical path into the wave guide and out of the wave guide.

The shifted focus of the first sensor and the second sensor (for example a different focal length of each sensor) may be realized by providing at least one additional lens in at least one of the optical paths of each sensor. However, only one additional lens may be provided in at least one optical path. Moreover, there may be provided further lenses or an optical system to appropriately direct light from the object to the sensors. Using the imaging system according to one embodiment of the present invention provides first and second image data that overlap perfectly with one another and that may be directly further processed without the need for any additional registration process. For example, perfectly overlapping images each having a different focus (e.g. focal point) may be used to determine depth information of the object (further details will follow below).

The optical channel may be an elongated body. Further, the optical channel may be hollow or made of a transparent material. The optical channel may have a proximal end facing the first sensor and the second sensor and a distal end facing the object to be imaged. The distal end may protrude from the imaging system and may be the outlet port of the first optical path and the second optical path. Therefore, the first optical path and second optical path are aligned to each other at the distal end such that the viewing angle of the first sensor and of the second sensor are the same. The optical channel may be configured such that the optical paths exiting the optical channel at the distal end extend in a divergent manner, preferably having an extension angle of 30° to 90°, preferably of 50° to 70° (i.e. the optical paths may become a cone shape once they have exited the optical channel so as to capture a larger scene). For example, the optical channel may be made of an optical fiber. In addition, the optical channel may be at least partly flexible such that it may guide light even if the optical channel describes a curve. Preferably, the optical channel is flexible in accordance to the shaft of a laparoscope in which it is provided. Therefore, objects positioned behind an obstacle may be easily imaged by routing the shaft around the obstacle. The imaging system may have only one optical channel. Accordingly, the imaging system may be compact such that only less space is necessary to operate the imaging system. On the other hand, stereoscopic imaging systems may have two or more optical channels due to inherent system requirements and therefore may need more space for operation.

The first and the second sensor may be photo sensors also referred to as image sensors or sensor chips, preferably complementary metal-oxide-semiconductor (CMOS) sensors, also known as complementary-symmetry metal-oxide-semiconductor (COS-MOS) sensors. Further, the first and the second sensor may be charge-coupled device (CCD) sensors. The first sensor and the second sensor may be the same kind of sensors. Alternatively, the first sensor may be a different sensor as compared to the second sensor. By providing different kind of sensors different image quality and/or different image information may be acquired. For example, a low-resolution image data may be used to provide rough information of the object, whereas a high-resolution image may be used in further processing and for detailed information. Preferably, the minimum resolution of at least one sensor is 1080×1920 pixels. Preferably, each sensor has the same resolution. Hereinafter, it is referred to the first sensor and to the second sensor, however, the imaging system may have more than two sensors, each sensor having its own optical path. That is, if three sensors are provided there are also three optical paths provided and so on. For example, the imaging system may have three, four, five or more sensors. In this case, each optical path of each sensor is guided at least partly through the same optical channel. The use of more sensors is specifically useful if the object to be imaged has a large spatial extension or if very detailed information of the object to be imaged are to be obtained. Further, each sensor may have its own shutter configured to control the amount of light that is applied onto the sensor. As a result, by adjusting the shutter speed, the sensor may be adapted to different light conditions. Alternatively, the sensor may acquire a continuous video stream of image data.

In case a CMOS sensor is used, the image data may be a voltage signal that is outputted by the sensor. Preferably, each pixel of the sensor may have a specific voltage signal. That is, CMOS sensor may output a digital signal. In case the CCD sensor is used, the image data may be a charge signal. The voltage signal is less prone to be deteriorated by electromagnetic fields and thus a CMOS sensor is preferably used as the sensor of the imaging system.

The image data may be an output of the sensors. Moreover, the image data may include brightness information for one or more color channels. A color channel may represent a specific light spectrum. For example, the image data may comprise information for the green color channel, the RGB color channel and/or the NIR (near-infrared) color channel. The RGB color channel is considered as including the green color channel, the red color channel and the blue color channel. Further, each sensor may provide image data including information of different color channels. That is, the first sensor may provide first image data including information of the green channel, whereas the second sensor may provide second image data including information of the NIR-channel. In addition, further combinations of different color channels of each sensor are possible such as NIR-NIR, RGB-RGB, green-green, NIR-RGB, green-NIR, green-RGB. Wherein each color channel may be defined by a different band of wavelengths. For further processing of the image data, the imaging system may be connected or connectable to a processing unit (e.g. a computer) or may have a control unit. The control unit may be configured to further process the image data. For example, the control unit may determine the information contained within the image data, e.g. a histogram showing the brightness distribution. Further, the control unit may be the same control unit configured to control the focusing means (refer to the above). In addition, the image data may comprise an image or photograph depicting the object. In order to cope with bad light conditions, the imaging system may have a light source (e.g., a lamp) configured to light the object to be imaged. In more detail, the light source may be coupled with an additional wave guide that is configured to guide the light of the light source within or parallel to the optical channel to the object to be imaged.

The optical paths are the paths that the rays of light take from the object to the sensors. That is, light may be reflected by the object to be imaged, introduced into the optical channel, guided through the optical channel, outputted by the optical channel and captured by the first and second sensors. The optical paths may be defined by rays of light that are received by the respective sensor. In other words, the length of the optical path may be measured from the sensor to the object to be imaged. In addition, within each optical path there may be provided lenses or arrays of lenses configured to appropriately guide the rays of light from the object to each of the sensors. Further, the imaging system may include at least one prism configured to split the first optical path and/or the second optical path. Preferably, the prism is configured as a beam splitter prism. The prism may be configured to filter a specific wavelength, i.e. to transfer only a specific wavelength of the light. Hence, the wavelength that is transmitted to the sensors may be pre-determined. For example, only the wavelength corresponding to the green channel may be transmitted by the prism. Further, the prism may also increase the length of one of the optical paths with respect to the other optical path. Accordingly, by the prism having several tasks, the imaging system may be realized with a minimum of components. In addition, the imaging system may include at least one aperture in the optical path or optical channel configured to control the aperture of the imaging system. That is, the wider the aperture is open, the shallower the depth of field of the image and vice versa. Generally, if the aperture is wide open, the image data has a better quality and includes more information. Therefore, it is preferred to adjust the aperture such that it has the maximum opening. As a result, the field of depth may relatively narrow. Further, the imaging system may have one aperture for each optical path.

The shifted focus of the first sensor and the second sensor may be provided by providing different focal lengths for each sensor. The focal length of the first sensor and the second sensor may be a distance from a main axis of an optical lens provided within the imaging system to the focus point along the respective optical path. The optical lens may be configured to project an image onto the sensor. Each sensor may be assigned to a separate lens. That is, in case there are two sensors, the imaging system may have two optical lenses. Further, the focal length may be a measure of how strongly the imaging system converges or diverges light. A positive focal length indicates that the system converges light, while a negative focal length indicates that the system diverges light. A system with a shorter focal length bends the rays more sharply, bringing them to a focus in a shorter distance or diverging them more quickly. For example, the ratio of the focus length of the first sensor and the second sensor may be in a range from 0.1 to 1, preferably in a range from 0.3 to 0.7. In this range the best imaging of the object may be archived. In other words, having the above ratio, the distance between the focus point of the first sensor and the focus point of the second sensor is in an optimal range. In more detail, if the first image data and the second image data are combined or compared to each other, the above ratio ensures that no information of the object is lost due to being completely out of focus. In other words, the ratio ensures that a distance between portions of the object that are in focus is not excessively large.

According to the present invention at least two image data may be received or generated that are registered perfectly with one another without any additional registration process. That is, image data of the object generated by the at least two sensors, may have the same size, the same location and may be imaged from the same view point. Accordingly, some portions of the object may be in focus on the first image data, while some other portions of the object may be in focus on the second image data. Further, some portions of the object may be partly in focus on the first image data and partly in focus on the second image data. Consequently, by combining or comparing both image data with each other an overall image or overall data may be generated that includes more information (e.g. more details, depth information etc.) of the object as compared to a single image of the object or a stereoscopic image of the object. Said combination or comparing of the image data is facilitated because both image data are registered with one another so as to overlap perfectly. That is, it is not necessary to register the image data with one another before the image data are further processed. In other words, the first image data and the second image data may include exactly the same portion (i.e. scene) of the object. As a result, the present invention provides a highly efficient way to generate image data that overlap perfectly and that can thus be easily processed. Preferably, the focus shift of the sensors is previously (i.e. prior to the imaging of the object) determined by the imaging system. That is, the focus shift may be set by the hardware of the imaging system.

Preferably, the first optical path and the second optical path have different lengths. Accordingly, the focus point of the first sensor is located at a different location as compared to the focus point of the second sensor. Preferably, the distance between at least one sensor and the proximal end of the optical channel and/or between the sensors may be adjustable. As a result, a focus distance of each sensor may be different, that is, the distance from the respective sensor to the point of the object that is sharply depicted by the respective image data. Providing different lengths of the optical paths is a simple and robust way to realize different focus distances of the first sensor and the second sensor (i.e. the focus shift of the first sensor and second sensor). For example, the sensors may have different distances from the proximal end of the optical channel. That is, the first sensor may be arranged within the imaging system so as to be located farther away from the proximal end of the optical channel as compared to the second sensor. As a result, the same sensors and the same image settings (e.g. aperture, shutter speed etc.) may be used, while the different focus points (i.e. the focus shift of the first sensor and the second sensor) are ensured by the different locations of the sensors within the imaging system. As a result, the same components may be used for acquiring the first image data and the second image data. Thus, the imaging system may be simplified and manufacturing costs may be lowered.

Preferably the first sensor and the second sensor are further configured to image the object simultaneously. That is, the first image data and the second image data may be generated or acquired at the same time. As a result, both the first image data and the second image data are registered perfectly with one another because there is no possibility that the object moves or changes its shape between the time when the first image data is acquired and when the second image data is acquired. That is, the first image data and the second image data may differ from each other only by their individual focus points (i.e. focus/blur). Therefore, the imaging system may include only one shutter used for both sensors provided within the imaging system. Hence, the system may be further simplified while at the same time a perfect overlapping of the first image data and second image data is ensured.

Preferably, the system further comprises a focus system arranged in the first optical path and/or the second optical path and configured to vary the focus of the first sensor and/or the second sensor. Accordingly, the focus (e.g. focus point or focal length) of at least one sensor may be adjusted. In other words, the distance between the focus point of the first sensor and/or the focus point of the second sensor may be changed. As a result, the imaging system may be adapted to different objects having different spatial extensions. Further, the imaging system may be adapted to different applications. For example, is case the imaging system is used during a laparoscopy (i.e. in combination with a laparoscope) the first sensor is focused at a distance of 6 cm measured from the distal end of the shaft and the second sensor may be focused at a distance of 9 cm measured from the distal end of the shaft. Therefore, the focus shift of the sensors is 3 cm. The focus shift may be positive or negative depending on the specific application. Hence the imaging system may be used in a plurality of applications and a plurality of objects may be imaged using the imaging system. For example, the focus system may be configured to adjust the focus point of at least one of the sensors such that the above defined ratio between the focus point of the first sensor and the focus point of the second sensor may be obtained.

Preferably, the system further comprises a focusing means configured to control the focus system such that the focus (e.g. the focal length) of the first sensor and/or the second sensor may be adjusted. The focusing means may be a focus ring configured to be grasped by a user's hand. Therefore, the focusing means may be exposed to the outside of the imaging system (and to the outside of the laparoscope) so as to be graspable by a user. A position (i.e. rotation) of the focus ring may depend on the distance of the object to be imaged from the sensors. The larger the distance to the object, the more the focus ring has to be rotated so as to depict at least a part of the object on one of the sensors in a sharp manner and vice versa. The focusing means may be sized such that only a few fingers (e.g. 2 fingers) of a user may be in contact therewith while the other fingers of a user's hand may hold the imaging system (i.e. laparoscope). As a result, the imaging system is favorable operatable using one hand. That is, a user does not need to operate the imaging system with both hands in order to adjust the focus and to hold the imaging system. As a result, the imaging system exhibits an improved operability. Moreover, the focusing means may be operated automatically by the control unit. In more detail, the focusing means may be controlled by the control unit so as to adjust the focus of each sensor depending of a specific application prior to the acquisition of the image data. Alternatively or additionally, the focusing means may be controlled by the control unit so as to adjust the focus distance of each sensor in predetermined steps (i.e. increments). At each step image data may be acquired by the first sensor and the second sensor. The step size may be in a range of 0.1 mm to 5 mm, preferably in a range of 0.5 mm to 2 mm. The focusing means may be provided at the camera head of the endoscope. Accordingly, at the shaft there may be no focusing means provided. This helps to keep the weight of the shaft low. As a result, the operability of the whole endoscope may be improved. Further, during operation of the endoscope, the focusing means be be positioned outside of the cavity under inspection (e.g. the human body). Therefore, the focusing means may be easily accessible for the user or for a robot.

Preferably, the first image data and the second image data represent an identical scene e.g. exactly the same portion of the object. Depending on the focus (e.g. the focal length) of the sensors and/or on different kind of sensors used, the field of view of the first image data may be smaller than the field of view of the second image data or vice versa. In order to provide the identical field of view of the object within the first image data and the second image data, the image data that covers a larger field of view may be adjusted so as to exactly include the same field of view of the object (e.g. by cutting away a part of the image data, e.g. the outer rim). As a result, both image data may be comparable or combinable in an easy way. For example, an edge of the image data may be used as a reference point in both image data. In other words, the part of the object represented by the first image data and the second image data overlap exactly. Hence, the further processing of the image data may be further improved and simplified.

Preferably, the imaging system further includes a control unit configured to generate depth information of the object based on the first image data and the second image data. In other words, the imaging system may include a control unit for further processing the image data. The control unit may be a device similar to a computer and may be configured to receive input data, to process the input data and to output processed data. In particular, the image data (e.g. a plurality of image data) including 2D coordinates of the object may be the input data and a third coordinate (i.e. the depth information or 3D shape information) of the object may be the output data. The generation of the depth information is one example for which the plurality of image data may be used. That is, the control unit may output a depth map (e.g. 3D image data) of the object. Specifically, the depth map may be a scatter plot of points each having three coordinates describing the spatial position or coordinate of each point of the object. That is, the control unit may be configured to determine a depth map based on the first image data and the second image data.

Further, the control unit may be configured to divide the image data into segments of one or several (e.g. >9) pixels, referred to as patches. In addition, the control unit may be configured to compare a patch of the first image data with a patch of the second image data. The patches may be rectangular or quadratic patches. In particular, the control unit may be configured to determine how sharp the object is depicted in each patch (i.e. to determine a sharpness of each patch). The patches may have multiple sizes. That is, the size of the patches provided in the respective image data may vary depending on the object that is depicted by the image data and/or on the specific application (e.g. the kind of surgery). That is, in areas where a lot of texture exists, the patch size may be smaller (e.g. 5×5 pixels), whereas in areas of the image data where pixel intensities are more homogeneous, i.e. there is little texture, the pixel size may be larger (e.g. 50×50 or up to 1000 pixels). For example, a lot of texture may be in an area of the image data where a lot of edges are depicted. Accordingly, the imaging system may work in a highly efficient way, because in areas where there is a lot of texture, the patch size is small (i.e. the resolution is high) so as to obtain highly accurate information of this area, whereas in areas where there is little texture, the patch size is bigger so as to accelerate the process carried out by the control unit.

Preferably, the location of the at least one or each first patch in the first image data corresponds to the location of the at least one or each second patch in the second image data. Preferably, the at least one first patch preferably has the same size as the at least one second patch, preferably a size of 20×20 pixels. That is, the first patch has the same location within the first image data as the second patch within the second image data. In more detail, if the first image data and the second image data are registered with one another, the first patch and the second patch overlap with one another. Accordingly, for each patch a depth value (i.e. the z-coordinate) may be determined by the control unit (i.e. for each patch x, y and z coordinates in 3D space are determined). A patch may have a size of one or several pixels. However, the required computing resources are depending on the number of patches included within the image data. Therefore, preferably each patch may have a size of 20×20 pixels. Having this size, the efficiency of the system is ensured while a high accuracy of the depth map is ensured.

Further, the control unit may be configured to determine the sharpness of each patch of the first image data and each patch of the second image data. It is to be noted that several patches may have the same sharpness. The sharpness of each patch is proportional to the entropy of the respective patch. In other words, the sharpness is synonymous for the entropy. A patch of the first image data may be considered as a first patch and a patch of the second image data may be considered as the second patch. The first patch and the second patch may overlap perfectly with one another (i.e. depicting the same scene/part of the object). In other words, the first patch and the second patch (also referred to as a pair of patches) may be located at the same location within the first image data and within the second image data. As explained above the focus distance of the first sensor and of the second sensor may be previously set. Therefore, the focus distances of the first sensor and of the second sensor is known. Using the following formula, the control unit may determine the depth (i.e. the z-coordinate) of the part of the object depicted by the first patch and the second patch.

$$d = \frac{( {d}_{1} * {I}_{1} + {d}_{2} * {I}_{2} )}{( {I}_{1} + {I}_{2} )}$$

wherein
d is the unknown distance (i.e. depth or z-coordinate) of a part of the object depicted in the first patch and the second patch,
d₁ is the focus distance of the first sensor,
I₁ is the sharpness of the first patch,
d₂ is the focus distance of the second sensor,
I₂ is the sharpness of the second patch.
   The focusing distances of the first sensor and the second sensor and the patch size has to be selected carefully so as to attain useful depth information using the above formula. Preferably, the focus distances are dependent on the specific application that is executed (e.g. the specific surgery or laparoscopy that is performed). For example, the focus distance of the first sensor may be 6 cm measured from the distal end of the shaft and the focus distance of the second sensor may be 9 cm from the distal end of the shaft. The focus distance of the first sensor may an empirical value determined from the type of surgery (e.g. for laparoscopy it may be 6 cm measured from the tip of the laparoscope (i.e. the distal end of the shaft)).

The sharpness of the image may be expressed by the information density of the image (e.g. the entropy) of the specific patch. The control unit may be configured to apply the above formula to each pair of patches (i.e. to each first patch and to each corresponding second patch) of the first image data and the second image data. As a result, a depth value for each pair of patches of the image data may be determined by the control unit. Having the depth information, the control unit may be configured to create a depth map using the depth information and the x and y coordinates of the respective pair of patches. In other words, the control unit may be configured to create a 3D model based on the x, y and z coordinates of each pair of patches.

Alternatively or additionally, the control unit may be configured to create a 3D model using the method of focus stacking. A further method that may be used to create a depth map is the so-called depth from focus/defocus. In addition, the control unit may be configured to carry out one or more of the above methods in order to create the depth information (i.e. 3D image data) based on at least two 2D image data (i.e. the first image data and the second image data). Specifically, the control unit may be configured to combine at least two methods for creating a depth map. In particular, the above formula may be applied to patches of the image data having a relatively high texture compared with other patches of the image data. In addition, the shape from lightning method may be applied to other regions of the image data having relatively poor texture compared with other regions of the image data. As a result, the performance of depth map generation may be significantly improved by applying different methods to different portions of the image data. Hence, at least one embodiment of the present invention may convert a relative difference of focus of the image data obtained by two sensors into 3D shape information. In other words, 3D coordinate of all patches within the image data may be calculated/determined by comparing the focus/blur of the respective patch (i.e. of one pixel or an array of pixels) generated by each sensor. Optionally, the control unit may be configured to further process the depth map by filtering the resulting depth map. In more detail, in order to delete erroneous depth information, the control unit may be configured to compare depth information of the depth map adjacent to each other. In case, one depth is excessively high or low (i.e. exceeding a preterminal threshold) compared to its neighbors, the control unit may be configured to delete such depth information because it is most likely that this depth information is an error. As a result, the outputted depth map by the control unit may have an increased accuracy.

Preferably, the control unit is further configured to generate the depth information by comparing the entropy of at least one first patch of the first image data and of at least one second patch of the second image data. The sharpness of the image is a synonym to the entropy. The entropy is an example of the information (i.e. a measure of the sharpness of a patch of the image data) included within the image data. That is, the first image data and the second image data may be compared to each other on the basis of their entropy. Accordingly, the control unit may be configured to determine the entropy of the first image data and of the second image data. Specifically, the higher the entropy, the more the respective image data is in focus. Accordingly, the lower the entropy, the less the respective image data is in focus (i.e. is blurred). The entropy may be determined by the following formula:

$$H = - {\sum }_{k} {p}_{k} {log}_{2} ( {p}_{k} )$$

wherein k is the number of levels of one channel or band (e.g. the gray-channel or the green-channel under observation) and pₖ is the probability associated with gray level k. Further, as the band the RGB-channel and/or the NIR-channel may be used. The sharpness I₁ and I₂ used in the above formula for determining the depth of a specific part of the object, may be respectively substituted by the entropy. That is, using the above formula, the entropy H₁ is determined for the first patch and the entropy H₂ is determined for the second patch (and for each further pair of patches). Then, H₁ and H₂ are used instead of the I₁ and I₂ in the formula for determining the depth of a specific part of the object depicted in the first patch and the second patch. That is, the entropy is synonymous for the sharpness.

In the following three optional features for improving the depth estimation accuracy are outlined:

Preferably, the control unit may be configured to monitor adjustments of the focus of at least one of the sensors. For example, the control unit may detect an operation of the focusing means. The focus may be adjusted by a human user and/or by a robot operating the imaging system. That is, if the focus of at least one of the sensors is adjusted the control unit may monitor the amount of adjustment. In other words, the control unit may detect a shift (i.e. a distance) of the focal point of each sensor. Based on the image data, the control unit may detect which part of the image data is sharply depicted (e.g. using edge detection). Further, the control unit may know what distance a sharply depicted portion of the object under examination is from the tip end of the endoscope, for example. By combining the knowledge of the position of the focal point within the image data and which part of the image data is sharply depicted, the control unit may improve the depth estimation. In other words, the control unit may gain information which part of the image data is sharply depicted at present and where the focus point of each sensor lies. As a result, the control unit may determine the depth of the sharply depicted portions of the image data. The monitoring may be conducted in real time. Accordingly, a continuous improvement of the depth estimation is possible. The monitoring of the focus adjustments may be realized by providing an optical encoder, electric measurements and/or other mechanical sensors. By using at least one of the above means the focus adjustments may be efficiently measured. In addition, the measuring accuracy may be increased by using a combination of these measuring means.

Preferably, the control unit may be configured to monitor movements of the imaging system. That is, the spatial position of the imaging system may be monitored by the control unit. For example, if the imaging system is provided in an endoscope, the control unit may detect the spatial position of the endoscope. Preferably, the position is detected in real time. This information may be then used to improve the depth estimation accuracy. The spatial position may be detected by optical trackers, electromagnetic trackers and/or by monitoring actuators. The actuators may be used to operate the imaging system by a robot. That is, if the robot moves the imaging system during operating it, the control unit may detect such movement and may thus determine the current spatial position of the imaging system. Further, the imaging system may include an acceleration senor and/or a rotation sensor. Thus, movements of the imaging system may be accurately monitored. Based on the known spatial position of the imaging system and information which part of the image data is sharply depicted (refer to the above outlined), the depth estimation accuracy may be further improved.

Preferably, the control unit may be configured to infer depth information using a size of known objects within the image data. That is, the control unit may be configured to determine a distance to a known object (e.g. from a tip end of the endoscope) by knowing the dimension of the object. The object may be markings present within the cavity under inspection. In addition, the markings may be provided on instruments additionally used during examination (e.g. during surgery). Further, the object may be an instrument itself or other objects present in the cavity under examination. Accordingly, an optical calibration may be executed in order to improve the depth estimation accuracy.

Improving the depth estimation accuracy may be useful in order to reduce wrong results of the depth estimation. Wrong results may sometimes occur due to the fact that the depth estimation may be based on contrast of portions of the first image data and the second image data (refer to the above outlined). The previously defined three features may be used to check whether the depth estimation is correct or not, for example. The latter feature may be advantageously because there is no additional structural means necessary to execute the optical calibration. In other words, the optical calibration may be implemented by the control unit without the need for any additional sensors etc. However, two or all three of the above features may be executed in order to improve the depth estimation accuracy. Moreover, the information obtained as outlined above may be also used to compensate distortion of the imaging system (e.g. of the endoscope comprising the imaging system). The at least one of the above features may be implemented in using the technique of computer vision. Computer vision may be an interdisciplinary scientific field that deals with how computers can gain high-level understanding from digital images or videos. From the perspective of engineering, it may seek to understand and automate tasks that the human visual system can do. Computer vision tasks may include methods for acquiring, processing, analyzing and understanding digital images, and extraction of high-dimensional data from the real world in order to produce numerical or symbolic information, e.g. in the forms of decisions. Understanding in this context may mean the transformation of visual images (image data) into descriptions of the world that make sense to thought processes and can elicit appropriate action. This image understanding may be seen as the disentangling of symbolic information from image data using models constructed with the aid of geometry, physics, statistics, and learning theory.

Preferably, the optical channel is rotatable such that a field of view of the first sensor and the second sensor is variable. In particular, only the distal end of the optical channel may be configured to be rotated. That is, the optical channel may be rotated only partly. The sensors may be fixed relative to the rotation of the optical channel. Alternatively, the optical device (e.g. a lens or an array of lenses) provided at a distal end of optical channel may be configured to vary the field of view by being rotated. Accordingly, the proximal end of the optical channel may be fixedly held within the imaging system while the distal end may be rotated so as to be directed towards an object to be imaged. The part of the optical channel that may be rotated, may protrude from the imaging system. The optical channel may have an initial position in which the optical channel extends straight without any curvature. In addition, the optical channel may be rotated about the axis of the optical channel in the initial position. Specifically, the optical channel may be configured such that the optical path may be inclined about 20° to 40°, preferably about 30° with respect to the axis of the optical channel in the initial position. Further, the optical paths may have a cone shape once the optical paths have left the optical channel. Said cone may be inclined by rotating at least a part of the optical channel about the central axis. Specifically, by being rotated, the optical paths may be deviated by 30° with respect to the central axis of the optical channel. Further, at least the distal end of the optical channel may be moveable with respect to the sensors. As a result, the imaging system may be used in a plurality of applications even in narrow spaces or cavities. That is, only a part of the optical channel may be directed towards the object to be imaged in order to acquire first and second image data. Accordingly, even objects difficult to access may be imaged by the imaging system. Further, by being able to rotate the field of view about the axis of the optical channel and by being able to incline the field of view with respect to the axis of the optical channel, an all-round view of the cavity under inspection may be provided. For example, an endoscope comprising the imaging system does not need to be moved within the cavity in order to attain an overall impression of the cavity.

Preferably, the first sensor and the second sensor are arranged such that they are inclined in relation to each other. That is, at least two of the sensors are inclined with respect to each other. For example, the sensors are inclined with respect to each other when their photo sensitive surfaces are inclined to each other. The inclination provides the effect that the imaging system may be more compact compared to a configuration in which the sensors a coplanar to each other. Preferably, the sensors are housed in a camara head which is separate from a shaft element configured to be inserted into a cavity under inspection. Accordingly, the camara head may have compact dimensions due to the arrangement of the sensors in an inclined manner. That is, an extension of the camera head along the direction of the shaft may be limited so as to ensure a compact camera head. In particular, the sensors may be inclined by substantially 90° with respect to each other. By substantially 90° may be meant an arrangement of the sensors forming an angle of 90° plus minus 10° between them. Accordingly, the imaging system may be produced efficiently, taking into account an acceptable level of tolerances.

Preferably, the first sensor and the second sensor are configured to use the RGB light spectrum, the green light spectrum and/or the NIR light spectrum to generate the first image data and the second image data. Therefore, depending on the specific application the light spectrum may be used that includes a lot of information under given conditions. Specifically, the green light spectrum has a lot of information under relatively poor light conditions. On the other hand, the NIR light spectrum provides a lot of information in almost dark light conditions. As a result, shapes and information may be easily recognized or determined by the image data under different light conditions. Further, the first sensor and the second sensor may use different light spectrums/channels. For example, the first sensor may use the green spectrum and the second sensor may use the NIR-spectrum. As a result, even under varying light conditions a depth information may be determined.

According to a further aspect of the present invention, a laparoscope comprising the above imaging system is provided. In other words, the laparoscope may be an endoscope including a camera head comprising the above imaging system. The camera head accommodating the imaging system may be connected to the laparoscope via a camera adapter. The focusing ring may be arranged on the camera adapter of the laparoscope so as bridge the camera head and a telescope of the laparoscope. The telescope may be a shaft of the laparoscope. The optical channel may be arranged within the shaft. Further, the shaft may be flexible so as to allow the optical channel to be rotated at a distal end of the laparoscope (e.g. the telescope) or may protrude from the distal end of the laparoscope. Further, the laparoscope may comprise a light source. The light source may be adjustable so as to illuminate an area to which the distal end of the optical channel is directed. Accordingly, the first and second image data may be appropriately generated even in bad light conditions. The light source may be preferably a LED light source. As a result, a laparoscope may be provided that may acquire at least two focus shifted image data using a single camera head. As a result, the laparoscope may have a highly integrated design and may be used in a wide field of applications. The laparoscope may be configured to be operated by a robot. That is, the laparoscope may have an interface configured to receive operation commands from the robot and to transmit information (e.g. image data) to the robot. Accordingly, the laparoscope can be used in an at least partially automated surgical environment.

According to a further aspect of the present invention a method for imaging an object is provided, wherein the method comprising: generating first image data of an object by imaging the object along a first optical path using a first sensor, and generating second image data of the object by imaging the object along a second optical path using a second sensor, wherein the first sensor and the second sensor are focus shifted, and wherein the first optical path and the second optical path are guided at least partly through the same optical channel.

Preferably, the method further comprises the step of dividing the first image data and the second image data into patches, wherein the patches of the first image data and the patches of the second image data correspond to each other. In other words, a patch of the first image data covers exactly the same area as the corresponding patch of the second image data. The corresponding patches of the first image data and of the second image data may be considered as a pair of patches.

Preferably, the method further comprising the step of comparing the first image data and the second image data with each other so as to generate depth information.

Preferably, the images are compared with one another by comparing at least one pair of patches.

Preferably, the first image data and the second image data are generated simultaneously.

In addition, the invention is also directed to a method of using the above endoscope. The method may comprise providing the above endoscope and rotating the shaft member more than 360° in one direction.

Accordingly, a region of interest may be holistically investigated by the 360° viewability. Further, the operability of the endoscope may be facilitated because a user may rotate the shaft member without the need to consider a stop or a limit in the rotational movement.

Further, the invention is also directed to a use of one of the above endoscopes as a borescope for a visual inspection of difficult-to-reach objects in a non-medical field. That is, borescopes may be used for visual inspection work where the target area is inaccessible by other means, or where accessibility may require destructive, time consuming and/or expensive dismounting activities. Borescopes may be used in nondestructive testing techniques for recognizing defects or imperfections. Further, borescopes may be used in the visual inspection of aircraft engines, aeroderivative industrial gas turbines, steam turbines, diesel engines, and automotive and truck engines. Gas and steam turbines require particular attention because of safety and maintenance requirements. Borescope inspection of engines can be used to prevent unnecessary maintenance, which can become extremely costly for large turbines. They are also used in manufacturing of machined or cast parts to inspect critical interior surfaces for burrs, surface finish or complete through-holes. In addition, the borescope may be used in forensic applications in law enforcement and building inspection, and in gunsmithing for inspecting the interior bore of a firearm. Alternatively, one of the above endoscopes may be used in the medical field. The endoscope may be used to visually examine and diagnose, or assist in surgery such as an arthroscopy. In particular, the endoscope may be used in a minimal invasive surgery (e.g. as a laparoscope). For example, the endoscope may be used to examine the internal organs like the throat or esophagus. In particular, the endoscope may be used to inspect human or animal organs like the bladder, the kidneys and the bronchus, or to inspect joints, the colon, the abdomen and the pelvis.

According to a further aspect of the present invention there may be provided an endoscope system comprising one of the above-described endoscopes, an external light source for transmitting light to the endoscope and a display device for displaying the image information transmitted by the endoscope.

According to a further aspect of the present invention there may be provided an endoscope system including an endoscope according to any one of the above configurations, and a trocar having a cannula configured to at least partly receive the endoscope such that the endoscope is moveable with respect to the trocar, wherein the cannula has a distal opening configured such that the endoscope may pass at least partly through the distal opening, wherein the cannula comprises a cleaning device provided at the distal opening and configured to clean at least the tip end of the shaft when the tip end passes the distal opening.

The trocar or trocar may be a medical or veterinary device that comprises an awl (which may be a metal or plastic sharpened or non-bladed tip), a cannula (essentially a hollow tube), and a seal. The trocars may be placed through the human's or animal's abdomen during laparoscopic surgery. The trocar may function as a portal for the subsequent placement of other instruments, such as graspers, scissors, staplers, endoscopes etc. The trocar may also allow the escape of gas or fluid from organs within the body.

The endoscope may be put into the cannula of the trocar. In particular, the shaft of the endoscope may be put into the cannula. In this state, the cannula and the shaft of the endoscope may be coaxial with each other. The cannula may extend in the axial direction. Further, the endoscope may be moved back and forth in the axial direction, wherein the canula may be fixedly held. In other words, the endoscope and the cannula may be positioned relative to each other such that the tip end of the endoscope and the tip opening of the cannula are aligned to each other. That is, the tip end of the endoscope and the tip opening of the cannula may be arranged essentially at the same position in the axial direction. For example, in this relative position the cleaning device may clean the tip end of the endoscope. The cleaning device may be similar in configuration to the cleaning device described above. In addition, the trocar may have the cleaning fluid supply device as described above. Accordingly, the endoscope may be configured in a compact size (i.e., without the cleaning device and/or cleaning fluid supply device), wherein the cleaning of the tip end of the endoscope is still possible by the trocar.

Individual features of the above defined embodiments may be rearranged or ex-changed with other features so as to form new embodiments. All advantages and modification of said features are analogously applicable to the new embodiments.

In the following the present invention will be explained in detail with reference to the enclosed figures. However, the detailed explanation of embodiments is provided for better understanding and is not intended to limit the scope of the present invention to the embodiments described in the following.

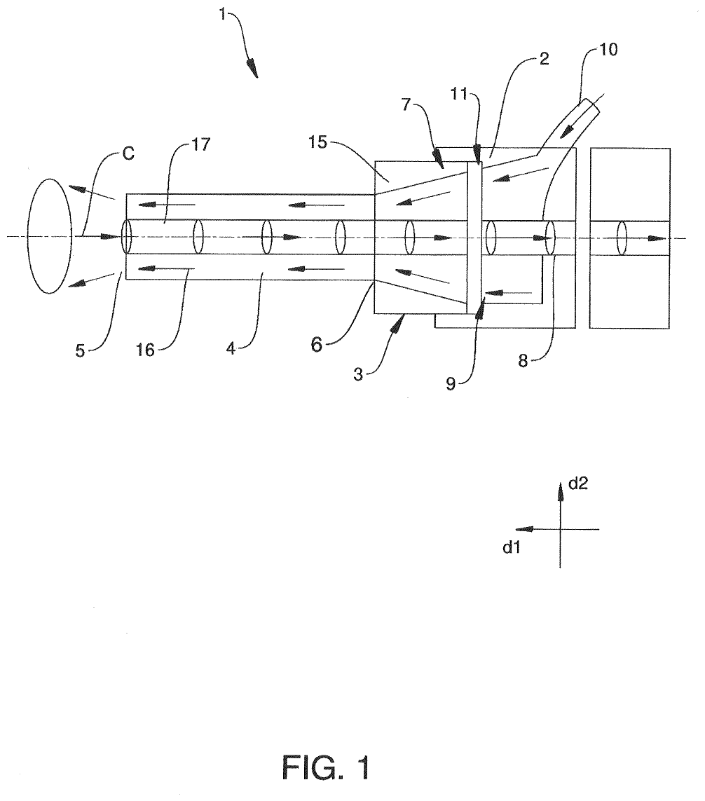

FIG. 1 is a schematic illustration of an endoscope according to an embodiment of the present invention.

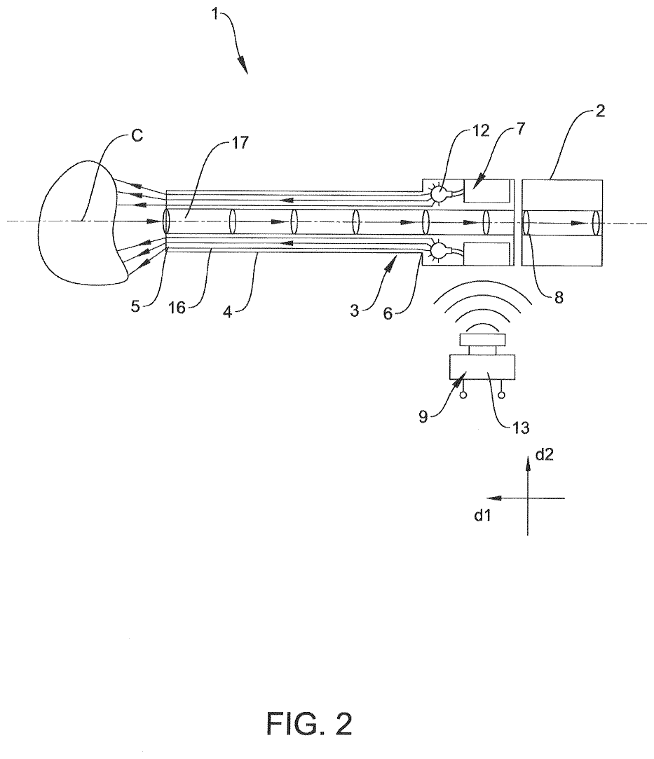

FIG. 2 is a schematic illustration of an endoscope according to an embodiment of the present invention.

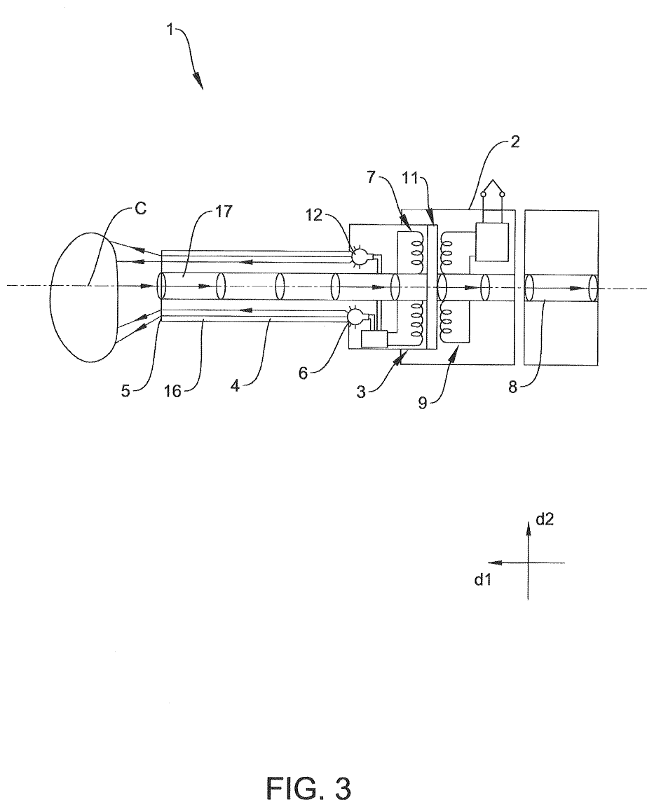

FIG. 3 is a schematic illustration of an endoscope according to an embodiment of the present invention.

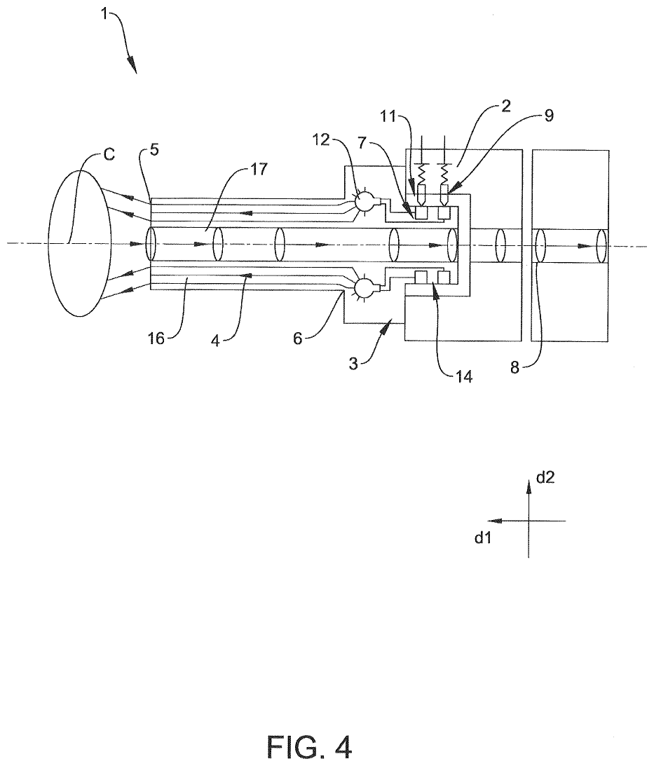

FIG. 4 is a schematic illustration of an endoscope according to an embodiment of the present invention.

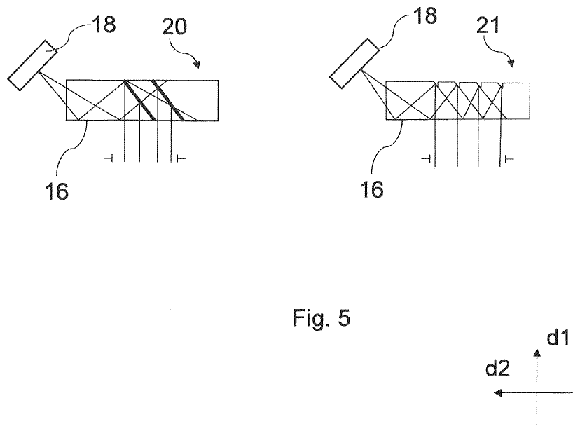

FIG. 5 is a schematic illustration of a light guide according to an embodiment of the present invention.

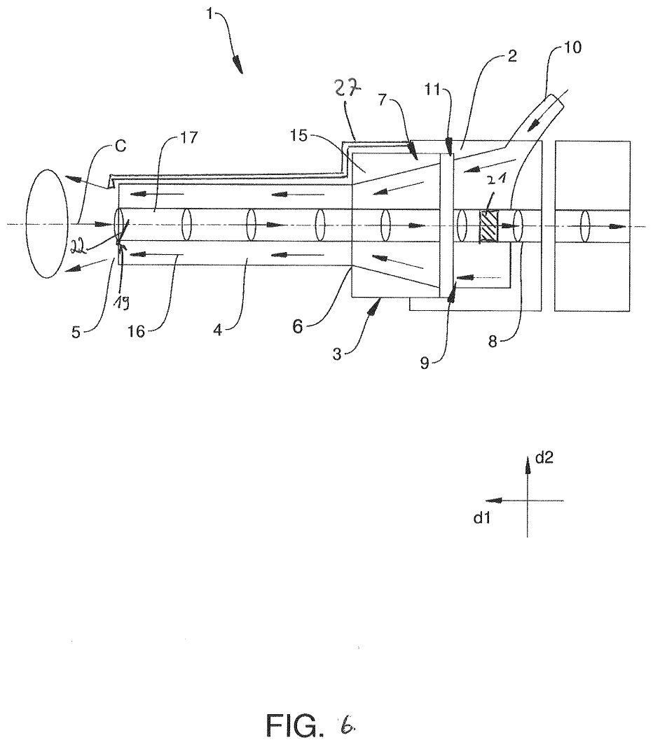

FIG. 6 is a schematic illustration of an endoscope according to an embodiment of the present invention.

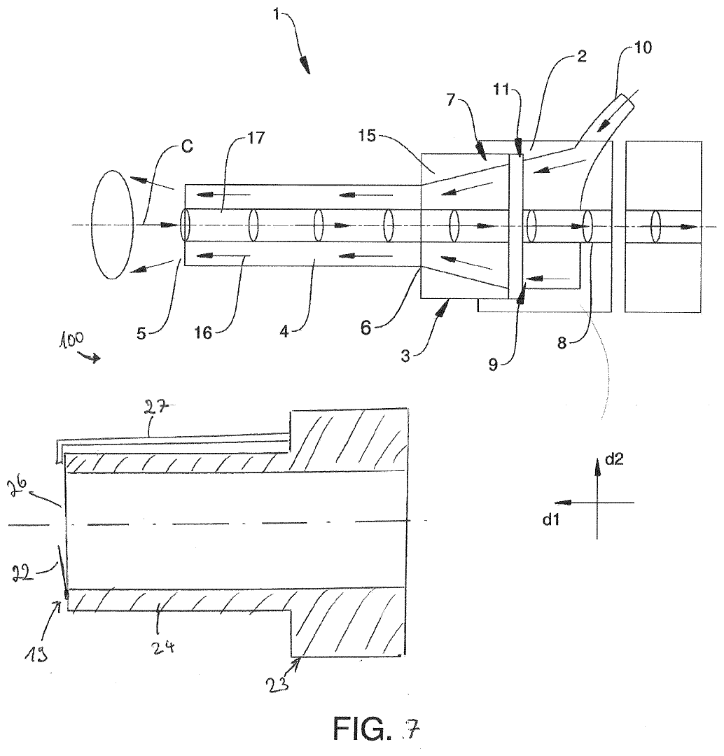

FIG. 7 is a schematic illustration of an endoscope system according to an embodiment of the present invention.

In the following, embodiments of the present invention will be described with regard to the enclosed figures. Similar features present in each embodiment are described only once and the description of similar features applies to each embodiment.

FIG. 1 is a schematic illustration of an endoscope 1 according to an embodiment of the present invention. In more detail, in FIG. 1 a schematic sectional view along the axial direction is depicted. The endoscope 1 includes a rotatable shaft member 3 and a fixed coupling member 2. The shaft member 3 includes a shaft 4 and a mounting base 15. The shaft 4 has an axial axis C extending along the axial directing d1. Further, the shaft 4 is a hollow member including a light guide 16 and an image guide 17. In particular, the image guide 17 is centered on the shaft axis C and is circumferentially surrounded by the light guide 16. The light guide 16 is configured to transmit light from a mounting end 6 of the shaft member 3 to a tip end 5 of the shaft member 3 (see arrows in the figures). Further, the image guide 17 is configured to transmit image information from the tip end 5 to the mounting end 6 (see arrows in the figures). At the mounting end 6 the shaft 4 is mounted to the mounting base 15.

The shaft member 3 is connected to a coupling member 2 so as to be unlimitedly rotatable with respect to the coupling member by 360° or more. That is, no stop that may limit the shaft member 3 from being infinitely rotated with respect to the coupling member 2 in one direction. The image information is transmitted from the shaft member 3 to the coupling member 2 along the axial direction d1. In particular, the image information is transmitted by the image guide 17 provided within the shaft member 3 and by an image information passage 8 provided within the coupling member 2. In some embodiments the image guide 17 and the image information passage 8 may be similarly configured. In addition, the light is transmitted from the coupling member 2 to the shaft member 3 in the axial direction d1, too, but in the opposite direction as compared to the image information. The light is transmitted by a transmitter 9 of the coupling member 2 to a receiver 7 of the shaft member 3. In the present embodiment the receiver 7 and the transmitter 9 are light transferring surfaces which are arranged opposite to each other sandwiching a gap 11 between them. In other words, the receiver 7 and the transmitter 9 are facing each other in the axial direction d1. The gap 11 is filled with a high transmission transparent liquid so as to reduce friction between the receiver 7 and the transmitter 9 when the shaft member 3 is rotated with respect to the coupling member 2 and to ensure a sufficient light transmission with reduced losses due to refraction. The light transferring surfaces forming the receiver 7 and the transmitter 9 have an annular shape around the extension of the shaft axis C. Further, the extension of the shaft axis C is their center. Accordingly, the image information passage 8 passes through the transmitter 9. In the same way the image guide 17 passes through the receiver 7. As a result, independent of the rotational position of the shaft member 3 and the coupling member 2 to each other, the light transfer from the coupling member 2 to the shaft member 3 and the image information transfer from the shaft member to the coupling member 2 is ensured. In other words, there is no mechanical connection between the shaft member 3 and the coupling member 2 for transferring either light from the coupling member 2 to the shaft member 3 or for transferring image information from the shaft member 3 to the coupling member 2. In some embodiments, the image information is also transmitted via the receiver 7 and the transmitter 9.

Moreover, the coupling member 2 has a port 10 for being connected with an external light source 18 (not depicted in FIG. 1). Accordingly, light may be inputted to the coupling member 2. The light may be distributed so as to be transferred to the transmitter 9. Further details will be explained with reference to FIG. 5 below.

Further, on the right side of FIG. 1 a coupling device is depicted. The coupling device comprises the image sensor to which the image information may be transmitted. In addition, the coupling device includes ports for connecting further processing devices and/or an eye piece. On the left side of FIG. 1 the object to be imaged is schematically depicted.

In a further embodiment not depicted in the figures, the coupling member 2 may have one or more light sources. That is, the light may be generated in the fixed part (i.e. the coupling member 2) and may be transferred via the transmitter 9 and the receiver 7 to the shaft member 3. In addition, the coupling member 2 may have a cooling arrangement so as to remove heat generated by the light source from the light source.

In a further embodiment, the endoscope 1 may comprise an imaging system (not separately depicted in the figures) which is configured to gather and to process image data. That is, the coupling member 2 and the shaft member 3 provide the mechanical handling part of the endoscope, whereas the imaging system provides the way of gathering or receiving the image data and the processing and evaluation thereof.

FIG. 2 is a schematic illustration of an endoscope 1 according to another embodiment of the present invention. The endoscope depicted in FIG. 2 differs from the above embodiment in that there is no light transmission between a rotating part (e.g. the shaft member 3) and a fixed part (e.g. the coupling member 2). In particular, the shaft member 3 comprises lamps (light source) 12 which are energized by electrical energy received by the receiver 7 from an external power supply or energy supply 13. In the present embodiment the external energy supply 13 is an inductive energy transmitter which is positioned near to the endoscope 1 such that the receiver 7 of the shaft member 3 may receive the energy transmitted by the inductive energy transmitter. Within the shaft member 3 a circuitry is provided so as to transform the energy in order to operate the lamps 12. The lamps 12 are connected to the light guide 16 of the shaft 4. The image information is transmitted from the shaft member 3 to the coupling member 2 in a contactless manner. Hence, the shaft member 3 may be readily rotatable with relative to the coupling member 2 in a continuous manner.

FIG. 3 is a schematic illustration of an endoscope 1 according to another embodiment of the present invention. The present embodiment differs from the embodiment depicted in FIG. 2 in that the receiver 7 and the transmitter 9 are formed as coils. That is, the receiver 7 is a power reception coil and the transmitter is a transmission coil. Both coils are arranged so as to face each other in the axial direction d1. In particular, the power reception coil and the transmission coil are circumferentially arranged about the extension of the shaft axis C. In the center of the coils the image information passage 8 and the image guide 17 pass through the respective coil. Accordingly, in each relative position of the shaft member 3 and the coupling member 2 to each other, energy may be transmitted to the shaft member 3 and image information may be transmitted to the coupling member 2. Similar as in the above embodiment, the shaft member 3 includes circuitry, in particular a rectifying circuit so as to transform the energy and to operate the lamps 12. In addition, the coupling member 2 includes a connection for power supply in order to supply electrical energy to the transmission coil.

FIG. 4 is a schematic illustration of an endoscope 1 according to another embodiment of the present invention. The present embodiment has a similar basic structure as the embodiments mentioned before. However, the present embodiment differs from the embodiment depicted in FIG. 3 in that the energy transmission is realized by brush contacts 14. That is, the shaft member 3 may have a contact portion formed as a cylindrical member. The contact portion has circumferential contacts on its outer surface. The circumferential contacts are provided circumferentially about the extension of the shaft axis C. The coupling member 2 has a reception corresponding to the shape of the contact portion of the shaft member 3. The reception is sized such that the contact portion may be accommodated within the receptacle. The receptacle has contacts biased by a spring such that the contacts are pressed against the circumferential contact of the contact portion. Accordingly, the electrical energy may be transmitted from the coupling member 2 to the shaft member 3 without hindering an unlimited rotation of the shaft member 3 relative to the coupling member 2. In a further embodiment the contact portion may be supported by the coupling member 2 via a bearing.

FIG. 5 is a schematic illustration of a light guide 16 according to an embodiment of the present invention. In particular, in FIG. 5 the part of the light guide 16 or of a distributor is depicted that is configured to redirect the light inputted into the endoscope 1 from an external light source 18. The light from the external light source 18 is inputted into the endoscope via the port 10. Accordingly, the light is inputted in the radial direction d2. Therefore, the light has to be redirected so as to be oriented in the axial direction d1. For this reason, the light guide 16 or the distributor may be configured to redirect the light from the radial direction d2 in the axial direction d1 and to evenly distribute the light such that it can be evenly transmitted by the transmitter 9. In this case the light distributor may be arranged within the coupling arrangement 2. In the following, two possibilities will be explained how to realize the redirection of the light. In each case depicted in FIG. 5, the light to be redirected comes from the left side and should be redirected towards the lower side of FIG. 5.

On the left side of FIG. 5 the redirection of the light is realized using a partially reflective mirror array 20. That is, partly reflective mirrors are provided in the cross section of the light guide. Accordingly, a part of the light is reflected in the desired direction (e.g. the axial direction d1). The other part of the light is transferred through the mirror and is then reflected by the next mirror of the mirror array. Accordingly, the redirected light has rays substantially parallel to each other and thus an evenly distributed light intensity.

On the right side of FIG. 5. the redirection of the light is realized using a micro-mirror array 21. That is, mirrors are provided so as to partly extend into the cross section of the light guide. Accordingly, only light is reflected that impinges one of the micro-mirrors is reflected. Similar to the above, the redirected light has rays substantially parallel to each other an evenly distributed light intensity.

In addition, a combination of both above defined redirection methods may be implemented so as to redirect the light. Further, the above-described redirection of light may be implemented for redirecting light that is inputted to the coupling member 2 from an external light source 18 using the distributor. In addition, the above-described redirection of light may be implemented for redirecting light at other positions of the endoscope 1. For example, the above-described redirection may be implemented at the tip end 5 of the shaft 4 so as to direct the light towards the object to be imaged.

FIG. 6 is a schematic illustration of an endoscope 1 according to another embodiment of the present invention. The present embodiment essentially corresponds to the embodiment depicted in FIG. 1 with the difference that in the present embodiment the endoscope comprises a cleaning device 19 and a cleaning fluid supply device 27. The cleaning device 19 comprises an elastic element 22 configured to wipe over the tip end 5 of the shaft 4. Accordingly, unwanted matter may be removed from the tip end of the shaft so as to ensure an undisturbed entering of the image information into the shaft 4. The elastic element 22 may be driven by a vibration. The vibration is induced to a prism provided at the tip end 5 of the shaft 4. In one embodiment, the cleaning device 19 includes a piezo electric element configured to apply the vibration to the elastic element 19 (e.g., via the prism). In order to support or to enforce the cleaning action of the cleaning device 19, there is provided a fluid supply device 27 configured to supply fluid to the tip end 5. The fluid supply device 27 is configured to eject the fluid towards the tip end 5 of the shaft. The fluid supply device 27 comprises a nozzle at its downstream end to accurately direct the fluid to the tip end 5.

In addition, the present embodiment comprises an adjusting means 28 to adjust the focus of the endoscope 1. The adjusting means 28 may be manually operated by a user via a focus ring and may be automatically operated by a computer via commands. The commands are issued by a control unit (not depicted in the figures) and are configured to bring a preselected point or subject in the image imaged by the endoscope in focus.

Any of the features of the present embodiment may be implemented solely in the embodiment shown in FIG. 1. FIG. 7 is a schematic illustration of an endoscope system 100 according to another embodiment of the present invention. The endoscope system 100 includes the endoscope 1 according to any of the previous embodiments and a trocar 23. The trocar has a cannula 24. The cannula 24 has a distal opening 26. The endoscope 1 may be inserted via its shaft 4 into the trocar 23. Then, the shaft 4 and the cannula 24 are coaxial with each other. The endoscope 1 and the trocar 23 may be moved with respect to each other in the axial direction d1. The trocar 23 has a cleaning device 19 provided at the tip opening 26 of the cannula 24. in case the tip end 5 of the shaft 4 passes the distal opening 26 of the trocar, the cleaning device 19 may be operated so as to clean the tip end 5 of the shaft 4. Further, the trocar has a cleaning fluid supply device 27 configured to supply fluid to the distal opening 26 of the trocar 23. The cleaning fluid may be ejected to the tip end 5 of the shaft any time the tip end 5 passes the distal opening 26. The cleaning device 19 and/or the cleaning fluid supply device 27 may be same as in the above embodiment.

### REFERENCE SIGNS

1 endoscope
2 coupling member
3 shaft member
4 shaft
5 tip end
6 mounting end
7 receiver
8 image information passage
9 transmitter
10 port
11 gap
12 light source
13 power supply
14 brush contacts
15 mounting base
16 light guide
17 image guide
18 external light source
19 cleaning device
20 reflective mirror array
21 micro mirror array
22 elastic element
23 trocar
24 cannula
26 distal opening
27 cleaning fluid supply device
28 adjustment means
100 endoscope system
C shaft axis
d1 axial direction
d2 radial direction

1. An endoscope, comprising:
a fixed coupling member, and
a rotatable shaft member including a shaft having a shaft axis extending in an axial direction, wherein the shaft has a tip end and a mounting end, wherein the shaft is configured to transmit light from the mounting end to the tip end and to transmit image information from the tip end to the mounting end,
wherein the fixed coupling member and the rotatable shaft member are connected to each other such that the rotatable shaft member is unlimitedly rotatable by 360° or more about the shaft axis relative to the fixed coupling member, and wherein the rotatable shaft member has a receiver configured to receive energy.

2. The endoscope of claim 1, wherein the fixed coupling member has an image information passage configured to transfer the image information from the rotatable shaft member in the axial direction.

3. The endoscope of claim 1, wherein the fixed coupling member has a transmitter configured to input the energy to the receiver in the axial direction or radial direction.

4. The endoscope of claim 1, wherein the fixed coupling member has at least one port for receiving light from a light source, and wherein the fixed coupling member is configured to transmit energy in the form of light to the receiver of the rotatable shaft member in the axial direction.

5. The endoscope of claim 4, wherein the at least one port is connected to the fixed coupling member such that the light is inputted into the fixed coupling member in a radial direction perpendicular to the axial direction, and wherein the fixed coupling member is configured to redirect the light so as to be transmitted to the receiver in the axial direction.

6. The endoscope of claim 1, wherein the fixed coupling member has a transmitter, wherein the transmitter of the fixed coupling member and the receiver of the rotatable shaft member are each formed as a light transfer surface, wherein the receiver and the transmitter are facing each other in the axial direction and are configured to transfer the light, wherein the receiver and the transmitter preferably have a circular shape.

7. The endoscope of claim 6, wherein the receiver and the transmitter are arranged circumferentially on an extension of the shaft axis and have a through hole for passing the image information passage through the receiver and the transmitter.

8. The endoscope of claim 6, wherein the transmitter and receiver are made of glass, transparent plastic or glass fiber.

9. The endoscope of claim 6, wherein the transmitter and the receiver are spaced apart from each other in the axial direction so as to form a gap between the receiver and the transmitter, wherein a transparent fluid is provided within the gap.

10. The endoscope of claim 1, wherein the rotatable shaft member includes at least one light source that is operated by electric energy received by the receiver.

11. The endoscope of claim 1, wherein the energy is inputted to the rotatable shaft member by a wireless power supply.

12. The endoscope of claim 1, wherein the fixed coupling member has a transmitter, wherein the transmitter of the fixed coupling member is formed as a transmission coil and the receiver of the rotatable shaft member is formed as receiving coil, wherein the receiver and the transmitter are facing each other in the axial direction and are configured to transfer the energy.

13. The endoscope of claim 1, wherein the energy is inputted to the rotatable shaft member via brush contacts between the rotatable shaft member and the fixed coupling member in the form of electrical energy.

14. The endoscope of claim 1, wherein the rotatable shaft member has different light sources each having a different wavelength.

15. The endoscope of claim 1, further comprising a cleaning device configured to clean the tip end of the shaft by wiping and/or vibrating.

16. The endoscope of claim 1, further comprising a cleaning fluid supply device configured to transfer a cleaning fluid and/or gas to the tip end of the shaft and to eject the cleaning fluid and/or gas towards the tip end of the shaft.

17. The endoscope of claim 1, wherein the fixed coupling member has an adjustment means for adjusting the focus of the endoscope automatically and/or manually.

18. The endoscope of claim 1, further comprising a time-of-flight sensor.

19. The endoscope of claim 1, wherein the fixed coupling member comprises an actuator for rotating the rotatable shaft member relative to the fixed coupling member.

20. The endoscope of claim 1, comprising an imaging system, wherein the imaging system comprises:
an optical channel configured to transfer light,
a first sensor configured to generate first image data by imaging an object along a first optical path, and
a second sensor configured to generate second image data by imaging the object along a second optical path,
wherein the first sensor and the second sensor are focus shifted, and
wherein the first optical path and the second optical path are guided at least partly through the optical channel.

21. The endoscope of claim 20, wherein the imaging system further includes a control unit configured to generate depth information of the object based on the first image data and the second image data.

22. The endoscope of claim 21, wherein the control unit is further configured to generate the depth information by comparing the entropy of at least one first patch of the first image data and of at least one second patch of the second image data, wherein the location of the at least one first patch in the first image data corresponds to the location of the at least one second patch in the second image data.

23. An endoscope system, comprising:
an endoscope comprising:
a fixed coupling member, and
a rotatable shaft member including a shaft having a shaft axis extending in an axial direction, wherein the shaft has a tip end and a mounting end, wherein the shaft is configured to transmit light from the mounting end to the tip end and to transmit image information from the tip end to the mounting end,
wherein the fixed coupling member and the rotatable shaft member are connected to each other such that the rotatable shaft member is unlimitedly rotatable about the shaft axis relative to the fixed coupling member, and wherein the rotatable shaft member has a receiver configured to receive energy, and
a trocar having a cannula configured to at least partly receive the endoscope such that the endoscope is moveable with respect to the trocar, wherein the cannula has a distal opening configured such that the endoscope may pass at least partly through the distal opening,
wherein the cannula comprises a cleaning device provided at the distal opening and configured to clean at least the tip end of the shaft d) when the tip end passes the distal opening.

24. A method of using an endoscope, the method comprising:
providing an endoscope comprising:
a fixed coupling member, and
a rotatable shaft member including a shaft having a shaft axis extending in an axial direction, wherein the shaft has a tip end and a mounting end, wherein the shaft is configured to transmit light from the mounting end to the tip end and to transmit image information from the tip end to the mounting end,
wherein the fixed coupling member and the rotatable shaft member are connected to each other such that the rotatable shaft member is unlimitedly rotatable about the shaft axis relative to the fixed coupling member, and wherein the rotatable shaft member has a receiver configured to receive energy, and
rotating the rotatable shaft member more than 360° in one direction relative to the fixed coupling member.
